(19) United States
(12) Patent Application Publication
(10) Pub. No.: US 2005/0214310 A1
(43) Pub. Date: Sep. 29, 2005

(54) Melphalan prodrugs

(75) Inventors: Brian E. Toki, Shoreline, WA (US); Peter D. Senter, Shoreline, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Seattle Genetics, Inc., Bothell, WA (US)

(21) Appl. No.: 11/043,428

(22) Filed: Jan. 24, 2005

Related U.S. Application Data
(60) Provisional application No. 60/538,790, filed on Jan. 23, 2004.

(51) Int. Cl. A61K 39/395; C12N 9/00

(52) U.S. Cl. 424/178.1; 435/188.5

(57) ABSTRACT

Shown and described are the synthesis of more potent forms of C-Mel, a prodrug used in Antibody-Directed Enzyme Prodrug Therapy, that releases the clinically used anticancer alkylating agent melphalan extracellularly. Shown and described are the synthesis of a variety of melphalan analogues with the intention to promote facile intracellular drug access. Esters, amides, and peptides of melphalan are shown. Cephalosporin prodrugs of the most interesting melphalan derivatives were synthesized and evaluated for potency, toxicity, therapeutic window, plasma stability, and solubility.

### REFERENCE TO EARLIER APPLICATION/PRIORITY CLAIM

This application claims the benefit of Provisional U.S. Application No. 60/538,790, filed Jan. 23, 2004, incorporated herein in its entirety by reference.

### BACKGROUND

The present invention is directed to novel cytotoxic agents and to a method for the delivery of novel cytotoxic agents to tumor cells. In particular, the invention is directed to prodrugs for the delivery of drugs to target cell populations, where the prodrugs are metabolized and activated by enzymes conjugated to targeting antibodies to provide active drugs.

In order to minimize toxicity problems, therapeutic agents are advantageously presented to patients in the form of prodrugs. Prodrugs are molecules capable of being converted to active therapeutic compounds in vivo by certain chemical or enzymatic modifications of their structure. For purposes of reducing toxicity, this conversion should be close to the site of action or target tissue rather than the circulatory system or non-target tissue. Since blood and serum contain enzymes which degrade, or activate, the prodrugs before the prodrugs reach the desired sites within the patient's body, prodrugs are often characterized by a low stability in blood and serum.

Antibody-Directed Enzyme Prodrug Therapy (ADEP™) utilizes monoclonal antibodies (mAbs) which bind to tumor-associated antigens. These mAbs are conjugated to enzymes capable of cleaving the prodrugs and releasing the active drugs at the tumor site. One advantage to this type of therapy is that it minimizes nonspecific uptake of the drug into normal cells.

U.S. Pat. No. 4,975,278, hereby incorporated by reference in its entirety, discusses a method for delivering cytotoxic agents to tumor cells by the combined use of antibody-enzyme conjugates and prodrugs. An enzyme that is capable of converting a poorly or non-cytotoxic prodrug into an active cytotoxic drug is conjugated to a tumor-specific antibody. This antibody-enzyme conjugate is administered to a tumor-bearing mammalian host and binds, due to the antibody specificity, to the surface of those tumor cells which possess the tumor antigen for which the antibody is specific. The prodrug is then administered to the host and is converted at the tumor site by the action of the antibody-bound enzyme into a more active cytotoxic drug.

Melphalan is one example of a nitrogen mustard and also known as L-PAM, phenylalanine mustard, L-sarcolysine, or 4[bis(2 chloroethyl)amino]-L-phynelalanine. Melphalan is a polar drug that has limited cell penetration. It is effective in the treatment of multiple myeloma, ovarian carcinoma, as adjuvant chemotherapy of stage II breast carcinoma, and in the regional perfusion of nonresectable melanoma, among other uses. Melphalan itself requires repeated high doses for clinical efficacy and undesirable side effects are generally seen. Most likely, the large doses are necessary due to its reduced uptake. The large neutral amino acid transporter is a known cellular trafficking system for melphalan, albeit with low affinity. Passive diffusion is considered poor due to melphalan's hydrophilic nature.

There is a need for melphalan prodrugs capable of use in ADEP™ that either improve these areas of intracellular access or create a new pathway for cell penetration. This would increase intratumoral concentrations thereby enhancing the potency. Furthermore, the prodrugs would need to have a plasma stability profile sufficiently long enough for prodrug to drug conversion followed by cellular uptake.

There is a need for melphalan prodrugs that are stable in circulation but can be cleaved intracellularly, where the released active drug would accumulate. Further, there is a need for melphalan prodrugs that have appropriate serum stability, yet is efficiently hydrolyzed inside a cell. There is also a need for melphalan prodrugs which may be transported into the cell through dipeptide transport systems or through increased drug hydrophobicity.

These and other limitations and problems of the past are solved by the present invention.

The recitation of any reference in this application is not an admission that the reference is prior art to this application.

### SUMMARY OF THE INVENTION

Methods for the treatment of cancer, an immune disorder, or an infectious disease and compounds effective for the treatment of cancer, an immune disorder, or an infectious disease are described herein.

In one embodiment, provided is a method for the delivery of cytotoxic agents to tumor cells including the administration of an effective amount of at least one antibody-enzyme conjugate comprising an antibody reactive with an antigen on the surface of the tumor cells conjugated to an enzyme which converts at least one prodrug having the formula 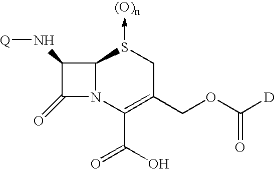
wherein
Q=H or salt thereof, C═O-alkyl, C═O-PEG, C═O-cycloalkyl, C═O-aryl, C═O-arylalkyl, CO₂R, or CONRR′ where R and R′ are, independently, an alkyl, alkenyl, alkynyl, heteroaryl alkyl, substituted alkyl, substituted aryl, substituted arylalkyl, heteroaryl, PEG, cycloalkyl, aryl, or arylalkyl;
n=0, 1, or 2;
   and D having the formula: 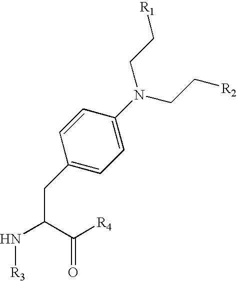
where R₁, R₂=independently halogens, O-mesylate, or O-tosylate,
R₃=H or lower alkyl groups C₁-C₆,
R₄=OH, PEG, alkoxy, cycloalkoxy, aryloxy, arylalkoxy, —NH₂, NHR, NRR′, where R and R′ are, independently, alkyl, alkenyl, alkynyl, heteroaryl alkyl, substituted alkyl, substituted aryl, substituted arylalkyl, heteroaryl, or is comprised of a peptide as follows:

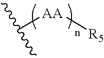

where AA is any given amino acid,

n=1 to 5 and
R₅ represents the manner in which the C-terminal amino acid or C-terminal amino acid derivative is capped at the carboxy terminus, if at all, and a pharmaceutically acceptable salt or solvent thereof.

In yet another embodiment, provided is a method for the prevention or treatment of cancer, an immune disorder, or an infection disease including the administration of an effective amount of a melphalan derivative having the formula: 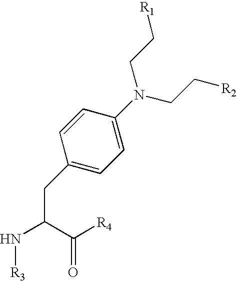
where R₁, R₂=independently halogens, O-mesylate, or O-tosylate,
R₃=H or lower alkyl groups C₁-C₆,
R₄=OH, PEG, alkoxy, cycloalkoxy, aryloxy, arylalkoxy, —NH₂, NHR, NRR′, where R and R′ are, independently, alkyl, alkenyl, alkynyl, heteroaryl alkyl, substituted alkyl, substituted aryl, substituted arylalkyl, heteroaryl, or is comprised of a peptide as follows:

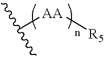

where AA is any given amino acid,

n=1 to 12 and
R₅ represents the manner in which the C-terminal amino acid or C-terminal amino acid derivative is capped at the carboxy terminus, if at all, and a pharmaceutically acceptable salt or solvent thereof.

In yet a further embodiment, provided is a method for the prevention or treatment of cancer, an immune disorder, or an infection disease including the administration of an effective amount of an antibody-drug conjugate including a targeting antibody conjugated to a melphalan derivative having the formula: 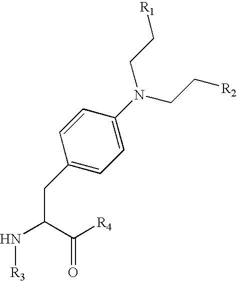
where R₁, R₂=independently halogens, O-mesylate, or O-tosylate,
R₃=H or lower alkyl groups C₁-C₆,
R₄=OH, PEG, alkoxy, cycloalkoxy, aryloxy, arylalkoxy, —NH₂, NHR, NRR′, where R and R′ are, independently, alkyl, alkenyl, alkynyl, heteroaryl alkyl, substituted alkyl, substituted aryl, substituted arylalkyl, heteroaryl, or is comprised of a peptide as follows:

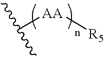

where AA is any given amino acid,

n=1 to 12 and
R₅ represents the manner in which the C-terminal amino acid or C-terminal amino acid derivative is capped at the carboxy terminus, if at all, and a pharmaceutically acceptable salt or solvent thereof.

In one embodiment, the present invention may be described as new prodrug compounds of a therapeutic agent, especially prodrugs comprising an antitumor therapeutic agent, displaying improved therapeutic properties relative to the products of the prior art, especially improved therapeutic properties in the treatment of cancerous tumors and/or in the treatment of inflammatory reactions. Improved therapeutic properties include decreased toxicity and increased efficacy. Particularly desired are prodrugs which display a high specificity of action, a reduced toxicity, an improved stability in the serum and blood, and which do not move into target cells until activated by a target cell associated enzyme.

In another embodiment, provided is a prodrug including an enzyme substrate portion and a drug portion, the drug portion having the formula: 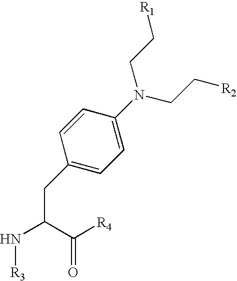
where R₁, R₂=independently halogens, O-mesylate, or O-tosylate,
R₃=H or lower alkyl groups C₁-C₆,
R₄=OH, PEG, alkoxy, cycloalkoxy, aryloxy, arylalkoxy, —NH₂, NHR, NRR′, where R and R′ are, independently, alkyl, alkenyl, alkynyl, heteroaryl alkyl, substituted alkyl, substituted aryl, substituted arylalkyl, heteroaryl, or is comprised of a peptide as follows:

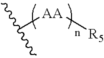

where AA is any given amino acid,

n=1 to 12 and
R₅ represents the manner in which the C-terminal amino acid or C-terminal amino acid derivative is capped at the carboxy terminus, if at all, and a pharmaceutically acceptable salt or solvent thereof.

In another embodiment, provided are derivatives of melphalan having the formula: 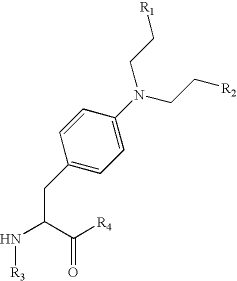
where R₁, R₂=independently halogens, O-mesylate, or O-tosylate,
R₃=H or lower alkyl groups C₁-C₆,
R₄=OH, PEG, alkoxy, cycloalkoxy, aryloxy, arylalkoxy, —NH₂, NHR, NRR′, where R and R′ are, independently, alkyl, alkenyl, alkynyl, heteroaryl alkyl, substituted alkyl, substituted aryl, substituted arylalkyl, heteroaryl, or is comprised of a peptide as follows:

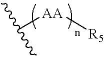

where AA is any given amino acid,

n=1 to 12 and
R₅ represents the manner in which the C-terminal amino acid or C-terminal amino acid derivative is capped at the carboxy terminus, if at all, and a pharmaceutically acceptable salt or solvent thereof.

In one aspect, the drug portion is melphalan and its derivatives. In another aspect, the melphalan derivatives provided include esters, amino acid esters, amino amides, D-amino acids, or other non-natural amino acids.

In yet a further embodiment, provided is a compound for the prevention or treatment of cancer, an immune disorder, or an infection disease including an antibody-drug conjugate including a targeting antibody conjugated to a melphalan derivative having the formula: 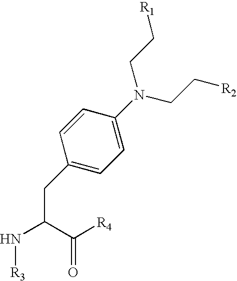
where R₁, R₂=independently halogens, O-mesylate, or O-tosylate,
R₃=H or lower alkyl groups C₁-C₆,
R₄=OH, PEG, alkoxy, cycloalkoxy, aryloxy, arylalkoxy, —NH₂, NHR, NRR′, where R and R′ are, independently, alkyl, alkenyl, alkynyl, heteroaryl alkyl, substituted alkyl, substituted aryl, substituted arylalkyl, heteroaryl, or is comprised of a peptide as follows:

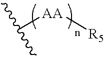

where AA is any given amino acid,

n=1 to 12 and
R₅ represents the manner in which the C-terminal amino acid or C-terminal amino acid derivative is capped at the carboxy terminus, if at all, and a pharmaceutically acceptable salt or solvent thereof.

In one aspect, a pharmaceutical composition is provided, such as one comprising a pharmaceutically effective amount of a prodrug having the formula 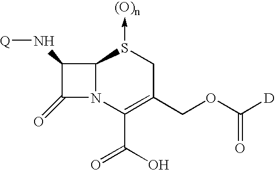
wherein
Q=H or salt thereof, C═O-alkyl, C═O-PEG, C═O-cycloalkyl, C═O-aryl, C═O-arylalkyl, CO₂R, or CONRR′ where R and R′ are, independently, an alkyl, alkenyl, alkynyl, heteroaryl alkyl, substituted alkyl, substituted aryl, substituted arylalkyl, heteroaryl, PEG, cycloalkyl, aryl, or arylalkyl;
n=0, 1, or 2;
   and D having the formula: 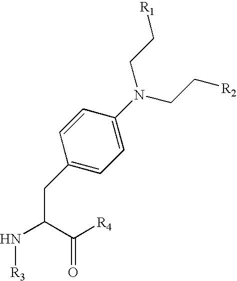
where R₁, R₂=independently halogens, O-mesylate, or O-tosylate,
R₃=H or lower alkyl groups C₁-C₆,
R₄=OH, PEG, alkoxy, cycloalkoxy, aryloxy, arylalkoxy, —NH₂, NHR, NRR′, where R and R′ are, independently, alkyl, alkenyl, alkynyl, heteroaryl alkyl, substituted alkyl, substituted aryl, substituted arylalkyl, heteroaryl, or is comprised of a peptide as follows:

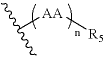

where AA is any given amino acid,

n=1 to 12 and
R₅ represents the manner in which the C-terminal amino acid or C-terminal amino acid derivative is capped at the carboxy terminus, if at all, and a pharmaceutically acceptable salt or solvent thereof, in admixture with a pharmaceutically acceptable carrier, diluent or excipient.

In another aspect, prodrug compounds of a marker enabling tumors to be characterized (diagnosis, progression of the tumor, assay of the factors secreted by tumor cells, etc.) are also contemplated. Thus, the invention includes a diagnosis or assay kit employing a compound of the invention.

The invention will best be understood by reference to the following detailed description of the preferred embodiment, taken in conjunction with the accompanying drawings. The discussion below is descriptive, illustrative and exemplary and is not to be taken as limiting the scope defined by any appended claims.

### BRIEF DESCRIPTION OF SEVERAL VIEWS OF THE DRAWINGS

FIG. 1 shows the structure of C-Mel and melphalan.

### DETAILED DESCRIPTION

When trade names are used herein, applicants intend to independently include the trade name product formulation, the generic drug, and the active pharmaceutical ingredient(s) of the trade name product.

Unless defined otherwise, all technical and scientific terms used herein have the same meaning as commonly understood by one of ordinary skill in the art pertinent to the methods and compositions described. The following references provide one of skill with a non-exclusive guide to a general definition of many of the terms used herein: Hale & Margham, The Harper Collins Dictionary of Biology (Harper Perennial, New York, N.Y., 1991); King & Stansfield, A Dictionary of Genetics (Oxford University Press, 4th ed. 1990); Hawley's Condensed Chemical Dictionary (John Wiley & Sons, 13th ed. 1997); and Stedmans' Medical Dictionary (Lippincott Williams & Wilkins, 27th ed. 2000). As used herein, the following terms and phrases have the meanings ascribed to them unless specified otherwise.

The term “antibody” herein is used in the broadest sense and specifically covers intact monoclonal antibodies, polyclonal antibodies, multispecific antibodies (e.g., bispecific antibodies) formed from at least two intact antibodies, and antibody fragments, so long as they exhibit the desired biological activity. An antibody is a protein generated by the immune system that is capable of recognizing and binding to a specific antigen. Described in terms of its structure, an antibody typically has a Y-shaped protein consisting of four amino acid chains, two heavy and two light. Each antibody has primarily two regions: a variable region and a constant region. The variable region, located on the ends of the arms of the Y, binds to and interacts with the target antigen. This variable region includes a complementary determining region (CDR) that recognizes and binds to a specific binding site on a particular antigen. The constant region, located on the tail of the Y, is recognized by and interacts with the immune system (Janeway, C., Travers, P., Walport, M., Shlomchik (2001) Immuno Biology, 5th Ed., Garland Publishing, New York). A target antigen generally has numerous binding sites, also called epitopes, recognized by CDRs on multiple antibodies. Each antibody that specifically binds to a different epitope has a different structure. Thus, one antigen may have more than one corresponding antibody.

The term “antibody” as used herein, also refers to a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species. In one aspect, however, the immunoglobulin is of human, murine, or rabbit origin. In another aspect, the antibodies are polyclonal, monoclonal, bispecific, human, humanized or chimeric antibodies, single chain antibodies, Fv, Fab fragments, F(ab′) fragments, F(ab′)₂ fragments, fragments produced by a Fab expression library, anti-idiotypic (anti-Id) antibodies, CDR's, and epitope-binding fragments of any of the above which immunospecifically bind to cancer cell antigens, viral antigens or microbial antigens.

The term “monoclonal antibody” as used herein refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see, U.S. Pat. No. 4,816,567). The “monoclonal antibodies” may also be isolated from phage antibody libraries using the techniques described in Clackson et al. (1991) Nature, 352:624-628 and Marks et al. (1991) J. Mol. Biol., 222:581-597, for example.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al. (1984) Proc. Natl. Acad. Sci. USA, 81:6851-6855).

Various methods have been employed to produce monoclonal antibodies (MAbs). Hybridoma technology, which refers to a cloned cell line that produces a single type of antibody, uses the cells of various species, including mice (murine), hamsters, rats, and humans. Another method to prepare MAbs uses genetic engineering including recombinant DNA techniques. Monoclonal antibodies made from these techniques include, among others, chimeric antibodies and humanized antibodies. A chimeric antibody combines DNA encoding regions from more than one type of species. For example, a chimeric antibody may derive the variable region from a mouse and the constant region from a human. A humanized antibody comes predominantly from a human, even though it contains nonhuman portions. Like a chimeric antibody, a humanized antibody may contain a completely human constant region. But unlike a chimeric antibody, the variable region may be partially derived from a human. The nonhuman, synthetic portions of a humanized antibody often come from CDRs in murine antibodies. In any event, these regions are crucial to allow the antibody to recognize and bind to a specific antigen.

As noted, murine antibodies can be used. While useful for diagnostics and short-term therapies, murine antibodies cannot be administered to people long-term without increasing the risk of a deleterious immunogenic response. This response, called Human Anti-Mouse Antibody (HAMA), occurs when a human immune system recognizes the murine antibody as foreign and attacks it. A HAMA response can cause toxic shock or even death.

Chimeric and humanized antibodies reduce the likelihood of a HAMA response by minimizing the nonhuman portions of administered antibodies. Furthermore, chimeric and humanized antibodies have the additional benefit of activating secondary human immune responses, such as antibody dependent cellular cytotoxicity.

“Antibody fragments” comprise a portion of an intact antibody, preferably comprising the antigen-binding or variable region thereof. Examples of antibody fragments include Fab, Fab′, F(ab′)₂, and Fv fragments; diabodies; linear antibodies; single-chain antibody molecules; and multispecific antibodies formed from antibody fragment(s).

An “intact” antibody is one which comprises an antigen-binding variable region as well as a light chain constant domain (CL) and heavy chain constant domains, CH1, CH2 and CH3. The constant domains may be native sequence constant domains (e.g., human native sequence constant domains) or amino acid sequence variant thereof.

The intact antibody may have one or more “effector functions” which refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g., B cell receptor; BCR), etc.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ∈, γ, and μ, respectively. The subunit structures and three-dimensional configurations of different classes of immunoglobulins are well known.

“Alkyl” is C₁-C₁₈ hydrocarbon containing normal, secondary, tertiary or cyclic carbon atoms. Examples are methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃.

“Alkenyl” is C₂-C₁₈ hydrocarbon containing normal, secondary, tertiary or cyclic carbon atoms with at least one site of unsaturation, i.e. a carbon-carbon, sp² double bond. Examples include, but are not limited to: ethylene or vinyl (—CH═CH₂), allyl (—CH₂CH═CH₂), cyclopentenyl (—C₅H₇), and 5-hexenyl (—CH₂CH₂CH₂CH₂CH═CH₂).

“Alkynyl” is C₂-C₁₈ hydrocarbon containing normal, secondary, tertiary or cyclic carbon atoms with at least one site of unsaturation, i.e. a carbon-carbon, sp triple bond. Examples include, but are not limited to: acetylenic (—C≡CH) and propargyl (—CH₂C≡CH).

“Aryl” means a monovalent aromatic hydrocarbon radical of 6-20 carbon atoms derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, naphthalene, anthracene, biphenyl, and the like.

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The arylalkyl group comprises 6 to 20 carbon atoms, e.g., the alkyl moiety, including alkanyl, alkenyl or alkynyl groups, of the arylalkyl group is 1 to 6 carbon atoms and the aryl moiety is 5 to 14 carbon atoms.

“Heteroarylalkyl” refers to an acyclic alkyl radical in which one of the hydrogen atoms bonded to a carbon atom, typically a terminal or sp³ carbon atom, is replaced with a heteroaryl radical. Typical heteroarylalkyl groups include, but are not limited to, 2-benzimidazolylmethyl, 2-furylethyl, and the like. The heteroarylalkyl group comprises 6 to 20 carbon atoms, e.g., the alkyl moiety, including alkanyl, alkenyl or alkynyl groups, of the heteroarylalkyl group is 1 to 6 carbon atoms and the heteroaryl moiety is 5 to 14 carbon atoms and 1 to 3 heteroatoms selected from N, O, P, and S. The heteroaryl moiety of the heteroarylalkyl group may be a monocycle having 3 to 7 ring members (2 to 6 carbon atoms or a bicycle having 7 to 10 ring members (4 to 9 carbon atoms and 1 to 3 heteroatoms selected from N, O, P, and S), for example: a bicyclo [4,5], [5,5], [5,6], or [6,6] system.

“Substituted alkyl”, “substituted aryl”, and “substituted arylalkyl” mean alkyl, aryl, and arylalkyl respectively, in which one or more hydrogen atoms are each independently replaced with a substituent. Typical substituents include, but are not limited to, —X, —R, —O⁻, —OR, —SR, —S⁻, —NR₂, —NR₃, ═NR, —CX₃, —CN, —OCN, —SCN, —N═C═O, —NCS, —NO, —NO₂, ═N₂, —N₃, NC(═O)R, —C(═O)R, —C(═O)NR₂, —SO₃⁻, —SO₃H, —S(═O)₂R, —OS(═O)₂OR, —S(═O)₂NR, —S(═O)R, —OP(═O)(OR)₂, —P(═O)(OR)₂, —PO₃⁻, —PO₃H₂, —C(═O)R, —C(═O)X, —C(═S)R, —CO₂R, —CO₂⁻, —C(═S)OR, —C(═O)SR, —C(═S)SR, —C(═O)NR₂, —C(═S)NR₂, —C(═NR)NR₂, where each X is independently a halogen: F, Cl, Br, or I; and each R is independently —H, C₂-C₁₈ alkyl, C₆-C₂₀ aryl, C₃-C₁₄ heterocycle, protecting group or prodrug moiety. Alkylene, alkenylene, and alkynylene groups as described above may also be similarly substituted.

“Heteroaryl” and “Heterocycle” refer to a ring system in which one or more ring atoms is a heteroatom, e.g., nitrogen, oxygen, and sulfur. The heterocycle radical comprises 1 to 20 carbon atoms and 1 to 3 heteroatoms selected from N, O, P, and S. A heterocycle may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo [4,5], [5,5], [5,6], or [6,6] system.

Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566.

Examples of heterocycles include by way of example and not limitation pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl.

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

“Carbocycle” means a saturated or unsaturated ring having 3 to 7 carbon atoms as a monocycle or 7 to 12 carbon atoms as a bicycle. Monocyclic carbocycles have 3 to 6 ring atoms, still more typically 5 or 6 ring atoms. Bicyclic carbocycles have 7 to 12 ring atoms, e.g., arranged as a bicyclo [4,5], [5,5], [5,6] or [6,6] system, or 9 or 10 ring atoms arranged as a bicyclo [5,6] or [6,6] system. Examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cycloheptyl, and cyclooctyl.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space. “Diastereomer” refers to a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

Examples of a “patient” or “subject” include, but are not limited to, a human, rat, mouse, guinea pig, monkey, pig, goat, cow, horse, dog, cat, bird and fowl. In an exemplary embodiment, the patient is a human.

“Aryl” refers to a carbocyclic aromatic group. Examples of aryl groups include, but are not limited to, phenyl, naphthyl and anthracenyl. A carbocyclic aromatic group or a heterocyclic aromatic group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

The term “C₁-C₈ alkyl,” as used herein refers to a straight chain or branched, saturated or unsaturated hydrocarbon having from 1 to 8 carbon atoms. Representative “C₁-C₈ alkyl” groups include, but are not limited to, -methyl, -ethyl, -n-propyl, -n-butyl, -n-pentyl, -n-hexyl, -n-heptyl, -n-octyl, -n-nonyl and -n-decyl; while branched C₁-C₈ alkyls include, but are not limited to, -isopropyl, -sec-butyl, -isobutyl, -tert-butyl, -isopentyl, 2-methylbutyl, unsaturated C₁-C₈ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, -3-methyl-1 butynyl, methyl, ethyl, propyl, isopropyl, n-butyl, isobutyl, sec-butyl, tert-butyl, n-pentyl, isopentyl, neopentyl, n-hexyl, isohexyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, n-heptyl, isoheptyl, n-octyl, and isooctyl. A C₁-C₈ alkyl group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —SO₃R′, —S(O)₂R′, —S(O)R′, —OH, -halogen, —N₃, —NH₂, —NH(R′), —N(R′)₂ and —CN; where each R′ is independently selected from H, —C₁-C₈ alkyl and aryl.

A “C₃-C₈ carbocycle” is a 3-, 4-, 5-, 6-, 7- or 8-membered saturated or unsaturated non-aromatic carbocyclic ring. Representative C₃-C₈ carbocycles include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. A C₃-C₈ carbocycle group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; where each R′ is independently selected from H, —C₁-C₈ alkyl and aryl.

A “C₃-C₈ carbocyclo” refers to a C₃-C₈ carbocycle group defined above wherein one of the carbocycle groups' hydrogen atoms is replaced with a bond.

A “C₁-C₁₀ alkylene” is a straight chain, saturated hydrocarbon group of the formula —(CH₂)₁₋₁₀—. Examples of a C₁-C₁₀ alkylene include methylene, ethylene, propylene, butylene, pentylene, hexylene, heptylene, ocytylene, nonylene and decalene.

A “C₃-C₈ heterocycle” refers to an aromatic or non-aromatic C₃-C₈ carbocycle in which one to four of the ring carbon atoms are independently replaced with a heteroatom from the group consisting of O, S and N. Representative examples of a C₃-C₈ heterocycle include, but are not limited to, benzofuranyl, benzothiophene, indolyl, benzopyrazolyl, coumarinyl, isoquinolinyl, pyrrolyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl and tetrazolyl. A C₃-C₈ heterocycle can be unsubstituted or substituted with up to seven groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

“C₃-C₈ heterocyclo” refers to a C₃-C₈ heterocycle group defined above wherein one of the heterocycle group's hydrogen atoms is replaced with a bond. A C₃-C₈ heterocyclo can be unsubstituted or substituted with up to six groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

The phrase “pharmaceutically acceptable salt,” as used herein, refers to pharmaceutically acceptable organic or inorganic salts. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

“Pharmaceutically acceptable solvate” or “solvate” refer to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine.

A “disorder” is any condition that would benefit from treatment of the present invention. This includes chronic and acute disorders or diseases including those pathological conditions which predispose the mammal to the disorder in question. Non-limiting examples of disorders to be treated herein include benign and malignant tumors; leukemia and lymphoid malignancies, in particular breast, ovarian, stomach, endometrial, salivary gland, lung, kidney, colon, thyroid, pancreatic, prostate or bladder cancer; neuronal, glial, astrocytal, hypothalamic and other glandular, macrophagal, epithelial, stromal and blastocoelic disorders; and inflammatory, angiogenic and immunologic disorders.

The term “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can, for example, be measured by assessing the time to disease progression (TTP) and/or determining the response rate (RR).

The term “substantial amount” refers to a majority, i.e. >50% of a population, of a collection or a sample.

The term “cytotoxic activity” refers to a cell-killing, cytostatic or anti-proliferation effect of an antibody drug conjugate compound or an intracellular metabolite of an antibody drug conjugate compound. Cytotoxic activity may be expressed as the IC₅₀ value which is the concentration (molar or mass) per unit volume at which half the cells survive.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

The terms “treat” or “treatment,” unless otherwise indicated by context, refer to both therapeutic treatment and prophylactic or preventative measures, wherein the object is to prevent or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

In the context of cancer, the term “treating” includes any or all of: preventing growth of tumor cells, cancer cells, or of a tumor; preventing replication of tumor cells or cancer cells, lessening of overall tumor burden or decreasing the number of cancerous cells, and ameliorating one or more symptoms associated with the disease.

In the context of an autoimmune disease, the term “treating” includes any or all of: preventing replication of cells associated with an autoimmune disease state including, but not limited to, cells that produce an autoimmune antibody, lessening the autoimmune-antibody burden and ameliorating one or more symptoms of an autoimmune disease.

In the context of an infectious disease, the term “treating” includes any or all of: preventing the growth, multiplication or replication of the pathogen that causes the infectious disease and ameliorating one or more symptoms of an infectious disease.

The term “acyl” refers to an organic radical derived from a carboxylic acid by the removal of the hydroxyl group.

The term “aromatic” refers to a cyclic conjugated compound with all or some of the atoms in the ring being carbons.

The term beta-lactams as used herein includes compounds having the general structure:

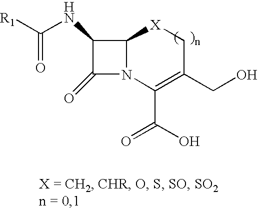

wherein R₁ includes, but is not limited to:

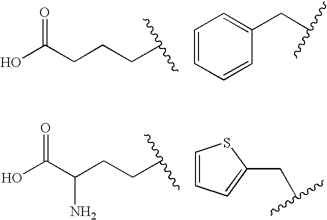

Beta-lactams include cephalosporins.

The term “beta-lactamase” as used herein refers to any enzyme capable of hydrolyzing the CO—N bond of a beta-lactam ring. The beta-lactamases are reviewed in Bush, Antimicrobial. Agents Chemother., 33:259, 1989.

The term “blocking group” refers to a chemical moiety that protects the reactive site that exists on the molecule described. Its purpose is to protect the atom or chemical moiety on the molecule of interest from undesired reactions via this reactive site. The blocking group(s) used during the synthesis of a prodrug described herein should remain attached to the site of interest until such a time is desired to remove the blocking group. The blocking groups suitable for protection of the amino group and carboxyl group are well known in the are of synthetic organic chemistry. Exemplary blocking groups for the amino group include, but are not limited to, Boc, Fmoc, trityl, 2-chlorotrityl, and 4-methoxytrityl. Exemplary blocking groups for the carboxyl group include, but are not limited to, methyl, ethyl, benzyl, diphenylmethyl, trityl, tert-butyl, and allyl.

As used herein, “cephalosporin” refers to derivatives of 7-aminocephalosporanic acid having the characteristic beta-lactam dihydrothiazine ring of cephalosporin C, occurring either naturally or synthetically. Cephalosporin is one of several broad spectrum antibiotic substances obtained from fungi and related to penicillin. The addition of side chains to cephalosporin has produced semisynthetic antibiotics. Examples of these derivatives and a review of the chemistry of the cephalosporins is given in Abraham, Quarterly reviews—Chemical Society, 21: 231, 1967.

Cephalosporins have the general structure:

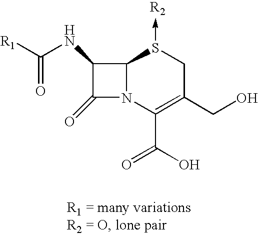

wherein R₁ includes, but is not limited to:

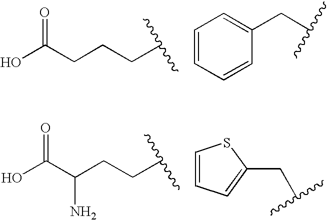

The term “cytotoxic” refers to arresting the growth of, or killing, cells.

The term “prodrug” refers to a compound that is relatively innocuous to cells while still in the prodrug form but which is selectively degraded to a pharmacologically active form by conditions, e.g., enzymes, located within or in the proximity of target cells.

The term “nitrogen mustard” as used herein refers to a compound of the general structure RN(CH₂CH₂Cl)₂, where R may be an alkyl, aryl, or aralkyl group substituted with a functional group amenable to further chemical modification, for example, an amino or a carboxyl group. Nitrogen mustards having more than one nitrogen atom are also included, such that both chloroethyl groups need not be attached to the same nitrogen atom. In some nitrogen mustards, the chlorine atoms may be replaced with other halogen atoms, especially bromine. Nitrogen mustards may also have suitable leaving groups such as a mesylate or tosylate that may replace the chlorine atoms. See, e.g., Stock, in Drug Design, E. J., Ariens, ed., Vol. II, pp. 532-571, Academic Press, New York, 1971.

For clarity of disclosure, and not by way of limitation, the detailed description of the invention is divided into the subsections which follow.

General Compounds

In one embodiment, the present invention includes new prodrug compounds capable of use as therapeutic agents in ADEPT™ or as diagnostic agents. ADEPT™ includes the administration of an effective amount of at least one antibody-enzyme conjugate of an antibody reactive with an antigen on the surface of the tumor cells conjugated to an enzyme which converts at least one prodrug to an active drug. The prodrug having the formula 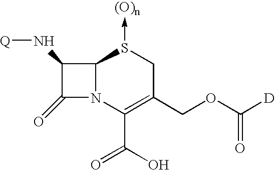
wherein
Q=H or salt thereof, C═O-alkyl, C═O-PEG, C═O-cycloalkyl, C═O-aryl, C═O-arylalkyl, CO₂R, or CONRR′ where R and R′ are, independently, an alkyl, alkenyl, alkynyl, heteroaryl alkyl, substituted alkyl, substituted aryl, substituted arylalkyl, heteroaryl, PEG, cycloalkyl, aryl, or arylalkyl;
n=0, 1, or 2;
   and D having the formula: 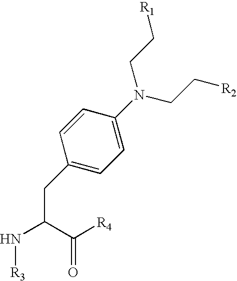
where R₁, R₂=independently halogens, O-mesylate, or O-tosylate,
R₃=H or lower alkyl groups C₁-C₆,
R₄=OH, PEG, alkoxy, cycloalkoxy, aryloxy, arylalkoxy, —NH₂, NHR, NRR′, where R and R′ are, independently, alkyl, alkenyl, alkynyl, heteroaryl alkyl, substituted alkyl, substituted aryl, substituted arylalkyl, heteroaryl, or is comprised of a peptide as follows:

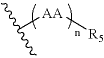

where AA is any given amino acid,

n=1 to 12 and

R₅ represents the manner in which the C-terminal amino acid or C-terminal amino acid derivative is capped at the carboxy terminus, if at all, and a pharmaceutically acceptable salt or solvent thereof. The amino acids include but are not limited to natural or synthetic, D or L, R or S, essential or non essential, alpha amino acids, beta amino acids, 3-amino acids, 4-amino acids, and 5-amino acids. Any amino acid AA includes but is not limited to those described herein and shown in the tables below:

| | One-Letter | Common |
|---|---|---|
| Amino Acid | Symbol | Abbreviation |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |
| β-alanine | | bAla |
| 2,3-diaminopropionic acid | | Dpr |
| β-aminoisobutyric acid | | Aib |
| N-methylglycine (sarcosine) | | MeGly |
| Ornithine | | Orn |
| Citrulline | | Cit |
| t-butylalanine | | t-BuA |
| t-butylglycine | | t-BuG |
| N-methylisoleucine | | MeIle |
| phenylglycine | | Phg |
| cyclohexylalanine | | Cha |
| norleucine | | Nle |
| naphthylalanine | | Nal |
| Pyridylananine | | |
| 3-benzothienyl alanine | | |
| 4-chlorophenylalanine | | Phe(4-Cl) |
| 2-fluorophenylalanine | | Phe(2-F) |
| 3-fluorophenylalanine | | Phe(3-F) |
| 4-fluorophenylalanine | | Phe(4-F) |
| Penicillamine | | Pen |
| 1,2,3,4-tetrahydro-isoquinoline- | | Tic |
| 3-carboxylic acid | | |
| ÿ-2-thienylalanine | | Thi |
| Methionine sulfoxide | | MSO |
| Homoarginine | | hArg |
| N-acetyl lysine | | AcLys |
| 2,4-diamino butyric acid | | Dbu |
| p-aminophenylalanine | | Phe(pNH₂) |
| N-methylvaline | | MeVal |
| Homocysteine | | hCys |
| Homoserine | | hSer |
| β-amino hexanoic acid | | Aha |
| β-amino valeric acid | | Ava |
| 2,3-diaminobutyric acid | | Dab |

The compounds that are encompassed within the scope of the invention are partially defined in terms of amino acid residues of designated classes. The amino acids may be generally categorized into two main classes: hydrophilic amino acids and hydrophobic amino acids, depending primarily on the characteristics of the amino acid side chain. These main classes may be further divided into subcategories that more distinctly define the characteristics of the amino acid side chains. For example, hydrophilic amino acids include amino acids having acidic, basic or polar side chains. Hydrophobic amino acids may include amino acids having aromatic or apolar side chains. They also may consist of amino acid esters, amino amides, and amino alcohols. Apolar amino acids may be further subdivided to include, among others, aliphatic amino acids. The definitions of the classes of amino acids as used herein are as follows:

“Hydrophobic Amino Acid” refers to an amino acid exhibiting a hydrophobicity of greater than zero according to the normalized consensus hydrophobicity scale of Eisenberg et al. (1984, J. Mol. Biol. 179: 125-142). Examples of genetically encoded hydrophobic amino acids include Pro, Phe, Trp, Met, Ala, Gly, Tyr, Ile, Leu and Val. Examples of non-genetically encoded hydrophobic amino acids include t-BuA, Glu-dimethyl ester, Phe-NH₂ (phenylalanine amide), phenylalaninol, homo-Phe-NH₂, Ser(OBz)-NH₂, propargyl-Ala-NH₂, and Gly-NHPh.

“Aromatic Amino Acid” refers to a hydrophobic amino acid having a side chain containing at least one aromatic or heteroaromatic ring. The aromatic or heteroaromatic ring may contain one or more substituents such as —OH, —SH, —CN, —F, —Cl, —Br, —I, —NO₂, —NO, —NH₂, —NHR, —NRR, —C(O)R, —C(O)OH, —C(O)OR, —C(O)NH₂, —C(O)NHR, —C(O)NRR and the like where each R is independently (C₁-C₆) alkyl, substituted (C₁-C₆) alkyl, (C₁-C₆) alkenyl, substituted (C₁-C₆) alkenyl, (C₁-C₆) alkynyl, substituted (C₁-C₆) alkynyl, (C₅-C₂₀) aryl, substituted (C₅-C₂₀) aryl, (C₆-C₂₆) alkaryl, substituted (C₆-C₂₆) alkaryl, 5-20 membered heteroaryl, substituted 5-20 membered heteroaryl, 6-26 membered alkheteroaryl or substituted 6-26 membered alkheteroaryl. Examples of genetically encoded aromatic amino acids include Phe, Tyr and Trp. Commonly encountered non-genetically encoded aromatic amino acids include phenylglycine, 2-naphthylalanine, ÿ-2-thienylalanine, 1,2,3,4-tetrahydroisoquinoline-3-carboxylic acid, 4-chloro-phenylalanine, 2-fluorophenylalanine, 3-fluorophenylalanine and 4-fluorophenylalanine.

“Apolar Amino Acid” refers to a hydrophobic amino acid having a side chain that is uncharged at physiological pH and which has bonds in which the pair of electrons shared in common by two atoms is generally held equally by each of the two atoms (i.e., the side chain is not polar). Examples of genetically encoded apolar amino acids include Gly, Leu, Val, Ile, Ala and Met. Examples of non-encoded apolar amino acids include Cha.

“Aliphatic Amino Acid” refers to a hydrophobic amino acid having an aliphatic hydrocarbon side chain. Examples of genetically encoded aliphatic amino acids include Ala, Leu, Val and Ile. Examples of non-encoded aliphatic amino acids include Nle.

“Hydrophilic Amino Acid” refers to an amino acid exhibiting a hydrophilicity of less than zero according to the normalized consensus hydrophobicity scale of Eisenberg et al. (1984, J. Mol. Biol. 179: 125-142). Examples of genetically encoded hydrophilic amino acids include Thr, His, Glu, Asn, Gln, Asp, Arg, Ser and Lys. Examples of non-encoded hydrophilic amino acids include Cet and hCys.

“Acidic Amino Acid” refers to a hydrophilic amino acid having a side chain pK value of less than 7. Acidic amino acids typically have negatively charged side chains at physiological pH due to loss of a hydrogen ion. Examples of genetically encoded acidic amino acids include Asp and Glu.

“Basic Amino Acid” refers to a hydrophilic amino acid having a side chain pK value of greater than 7. Basic amino acids typically have positively charged side chains at physiological pH due to association with hydronium ion. Examples of genetically encoded basic amino acids include Arg, Lys and His. Examples of non-genetically encoded basic amino acids include the non-cyclic amino acids omithine, 2,3-diaminopropionic acid, 2,4-diaminobutyric acid and homoarginine.

“Polar Amino Acid” refers to a hydrophilic amino acid having a side chain that is uncharged at physiological pH, but which has one bond in which the pair of electrons shared in common by two atoms is held more closely by one of the atoms. Examples of genetically encoded polar amino acids include Ser, Thr, Asn and Gln. Examples of non-genetically encoded polar amino acids include citrulline, N-acetyl lysine and methionine sulfoxide.

The amino acid residue Cys is unusual in that it can form disulfide bridges with other Cys residues or other sulfanyl-containing amino acids. The ability of Cys residues (and other amino acids with —SH containing side chains) to exist in a peptide in either the reduced free —SH or oxidized disulfide-bridged form affects whether Cys residues contribute net hydrophilic or hydrophobic character to a peptide. While Cys exhibits hydrophobicity of 0.29 according to the normalized consensus scale of Eisenberg et al. (supra), it is understood that Cys is classified as a polar hydrophilic amino acid for the purpose of the present invention. Typically, cysteine-like amino acids generally have a side chain containing at least one thiol (SH) group. Examples of genetically encoded cysteine-like amino acids include Cys. Examples of non-genetically encoded cysteine-like amino acids include homocysteine and penicillamine.

As will be appreciated by those having skill in the art, the above classifications are not absolute—several amino acids exhibit more than one characteristic property, and can therefore be included in more than one category. For example, tyrosine has both an aromatic ring and a polar hydroxyl group. Thus, tyrosine has dual properties and can be included in both the aromatic and polar categories. Similarly, in addition to being able to form disulfide linkages, cysteine also has apolar character. Thus, while not strictly classified as a hydrophobic or apolar amino acid, in many instances cysteine can be used to confer hydrophobicity to a peptide.

Certain commonly encountered amino acids which are not genetically encoded of which the peptides and peptide analogues of the invention may be composed include, but are not limited to, ÿ-alanine (b-Ala) and other omega-amino acids such as 3-aminopropionic acid (Dap), 2,3-diaminopropionic acid (Dpr), 4-aminobutyric acid and so forth; ÿ-aminoisobutyric acid (Aib); ÿ-aminohexanoic acid (Aha); ÿ-aminovaleric acid (Ava); N-methylglycine or sarcosine (MeGly); ornithine (Om); citrulline (Cit); t-butylalanine (t-BuA); t-butylglycine (t-BuG); N-methylisoleucine (MeIle); phenylglycine (Phg); cyclohexylalanine (Cha); norleucine (Nle); 2-naphthylalanine (2-Nal); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); ÿ-2-thienylalanine (Thi); methionine sulfoxide (MSO); homoarginine (hArg); N-acetyl lysine (AcLys); 2,3-diaminobutyric acid (Dab); 2,4-diaminobutyric acid (Dbu); p-aminophenylalanine (Phe(pNH₂)); N-methyl valine (MeVal); homocysteine (hCys) and homoserine (hSer). These amino acids also fall conveniently into the categories defined above.

The classifications of the above-described genetically encoded and non-encoded amino acids are summarized in Table A, below. It is to be understood that Table A is for illustrative purposes only and does not purport to be an exhaustive list of amino acid residues which may comprise the peptides and peptide analogues described herein. Other amino acid residues which are useful for making the peptides and peptide analogues described herein can be found, e.g., in Fasman, 1989, CRC Practical Handbook of Biochemistry and Molecular Biology, CRC Press, Inc., and the references cited therein. Amino acids not specifically mentioned herein can be conveniently classified into the above-described categories on the basis of known behavior and/or their characteristic chemical and/or physical properties as compared with amino acids specifically identified.

TABLE A

| Classification | Genetically Encoded | Genetically Non-Encoded |
|---|---|---|
| Hydrophobic | | |
| Aromatic | F, Y, W | Phg, Nal, Thi, Tic, Phe(4-Cl), |
| | | Phe(2-F), Phe(3-F), Phe(4-F), |
| | | Pyridyl Ala, Benzothienyl Ala |
| Apolar | L, V, I, A, M, G, P | T-BuA, T-BuG, MeIRe, Nle, |
| | | MeVal, Cha, MeGly, Aib |
| Aliphatic | A, V, L, I | t-BuA, t-BuG, MeIle, Nle, MeVal, |
| | | Cha, bAla, MeGly, Aib, Dpr, Aha |
| Hydrophilic | | |
| Acidic | D, E | |
| Basic | H, K, R | Dpr, Orn, hArg, Phe(p-NH₂), Dbu, |
| | | Dab |
| Polar | C, Q, N, S, T | Cit, AcLys, MSO, hSer, bAla |
| Helix-Breaking | P, G | D-Pro and other D-amino acids (in |
| | | L-peptides) |

In one aspect, the drug unit D of the prodrug is a nitrogen mustard compound including but are not limited to chlorambucil (CA), phenylacetic mustard (PDM), phynelproprionic mustard, and melphalan. In another aspect, the drug unit is melphalan and its derivatives. The melphalan derivatives provided include, but are not limited to, esters, amides, amino acid esters, amino amides, D-amino acids, D-amino amides or other non-natural amino acids.

The prodrugs are associated with a beta-lactam, such as but not limited to, cephalosporin.

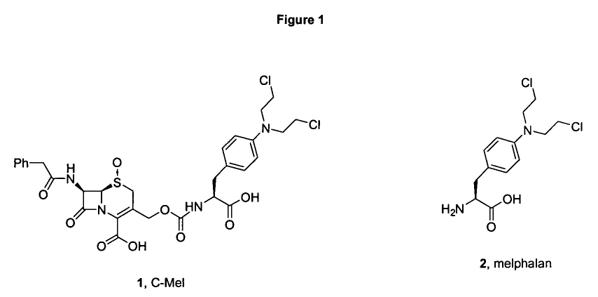

In another aspect, the prodrug includes an enzyme substrate portion other than a beta-lactam. Enzymes, such as but not limited to beta-lactamase, are effective in activation of a variety of anticancer prodrugs. Cephalosporin-type prodrugs such as but not limited to celphalosporin-nitrogen mustard drugs, such as but not limited to C-Mel 1, as shown in FIG. 1, are activated by beta-lactam hydrolysis which induces an electronic cascade to release melphalan 2, as shown in FIG. 1, near the tumor site.

The prodrugs of this invention are not limited to these compounds, and may include other antitumor agents that can be derivatized into a prodrug form and associated with a beta lactam. Such antitumor agents include etoposide, teniposide, daunomycin, carminomycin, aminopterin, dactinomycin, cis-platinum and cis-platinum analogues, bleomycins, esperamicins and 5-fluorouracil. A description of the synthesis of various of these drugs is described in U.S. Pat. No. 5,773,435, incorporated herein in its entirety.

The prodrugs of this invention include, but are not limited to, substrate portions, e.g., phosphate-containing prodrugs, thiophosphate-containing prodrugs, sulfate-containing prodrugs, peptide-containing prodrugs, D-amino acid-modified prodrugs, glycosylated prodrugs, optionally substituted phenoxyacetamide-containing prodrugs or optionally substituted phenylacetamide-containing prodrugs, 5-fluorocytosine and other 5-fluorouridine prodrugs which can be converted by the enzyme of the conjugate into the more active, cytotoxic free drug.

Enzymes that are useful to act upon the prodrugs include, but are not limited to alkaline phosphatase useful for converting phosphate-containing prodrugs into free drugs, arylsulfatase useful for converting sulfate-containing prodrugs into free drugs, cytosine deaminase useful for converting non-toxic 5-fluorocytosine into the anti-cancer drug, 5-fluorouracil, proteases, such as serratia protease, thermolysin, subtilisin, carboxypeptidases and cathepsins (such as cathepsins B and L), that are useful for converting peptide-containing prodrugs into free drugs, D-alanylcarboxypeptidases, useful for converting prodrugs that contain D-amino acid substituents, carbohydrate-cleaving enzymes such as beta-galactosidase and neuraminidase useful for converting glycosylated prodrugs into free drugs, and pencillin amidases, such as pencillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as abzymes, can be used to convert the prodrugs of the invention into free active drugs (see, e.g., R. J. Massey, Nature, 328, pp. 457-458 (1987)).

In another embodiment, provided are nitrogen mustard compounds having the formula: 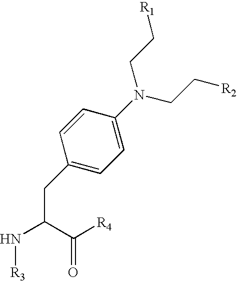
where R₁, R₂=independently halogens, O-mesylate, or O-tosylate,
R₃=H or lower alkyl groups C₁-C₆,
R₄=OH, PEG, alkoxy, cycloalkoxy, aryloxy, arylalkoxy, —NH₂, NHR, NRR′, where R and R′ are, independently, alkyl, alkenyl, alkynyl, heteroaryl alkyl, substituted alkyl, substituted aryl, substituted arylalkyl, heteroaryl, or is comprised of a peptide as follows:

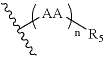

where AA is any given amino acid,

n=1 to 12 and
R₅ represents the manner in which the C-terminal amino acid or C-terminal amino acid derivative is capped at the carboxy terminus, if at all, and a pharmaceutically acceptable salt or solvent thereof. The amino acids include but are not limited to natural or synthetic, D or L, R or S, essential or non essential, and alpha amino acids.

In one aspect, the nitrogen mustard compounds include but are not limited to chlorambucil (CA), phenylacetic mustard (PDM), phynelproprionic mustard, and melphalan. In one aspect, the melphalan derivatives provided include, but are not limited to, esters, amino acid esters, amino amides, D-amino acids, or other non-natural amino acids.

Ester and amide derivatives of melphalan have certain different properties. The esters derivatives of melphalan are at least 10-fold more potent than melphalan. The half life of ester derivatives is effected by ester hydrolysis which leads to melphalan generation. Whereas, the amino acid amide derivatives of melphalan will not be rapidly hydrolyzed by esterases.

Esters and amides derivatives of melphalan provide a manner by which to increase, for example, the hydrophobicity of melphalan while still leaving it amenable for substrate association. Another approach is through melphalan peptides. Not only can the hydrophobic character of melphalan be increased, the methods of intracellular uptake can include peptide transport systems.

These types of melphalan modifications result in a more hydrophobic prodrug that could be problematic for formulation and injection purposes, especially at high doses. To circumvent these potential shortcomings, the hydrophobic phenacetyl group at the C-7 position on the cephalosporin was replaced with a glutaryl moiety. This in effect reintroduces the carboxyl group lost in the manipulation of melphalan and therefore promotes solubility characteristics similar to C-Mel 1, as shown in FIG. 1. Cephalosporin retains its sensitivity to beta-lactamase when bearing the glutaryl functionality at C-7. Vrudhula, V. M., et al. Bioconjugate Chem. (1993), 4, pp. 334-340.

General Methods

Provided are methods for the prevention and treatment of cancer, immune disorders, and infectious diseases. A novel method for the delivery of cytotoxic agents to tumor cells and providing for enhanced selective killing of tumor cells in the treatment of cancers, such as carcinomas and melanomas, as well as other tumors, is shown and described.

According to one aspect of the method, a targeting moiety, such as but not limited to a ligand, peptide, or antibody, conjugated to an enzyme is administered to a tumor-bearing mammalian host. In a particular aspect, the targeting moiety is an antibody-enzyme conjugate. This antibody-enzyme conjugate consists of a tumor-selective antibody linked to a beta-lactamase that is capable of converting a prodrug that is less cytotoxic to cells than the parent drug into the more active parent drug. When introduced into the host, the antibody component of the conjugate, which is reactive with an antigen found on the tumor cells, directs the conjugate to the site of the tumor and binds to the tumor cells. The antibody thus delivers the enzyme to the site of the tumor. A prodrug that is a substrate for the beta-lactamase is also introduced into the host and is converted, at the tumor site, by the enzyme into an active cytotoxic drug. The drug is thus activated extracellularly and can diffuse into all of the tumor cells at that site. The method therefore overcomes the current problems of tumor antigen heterogeneity and the requirement of antigen/conjugate internalization associated with conventional immunoconjugate drug delivery techniques.

Furthermore, because the present method does not require the drug to be bound directly to the antibody and thereby limit the amount of drug that can be delivered, the common-place problem of drug potency at the tumor site does not arise. In fact, the present method amplifies the number of active drug molecules present at the tumor site because the antibody-bound enzyme of the conjugate can undergo numerous substrate turnovers, repeatedly converting prodrug into active drug. Moreover, the present method is capable of releasing the active drug specifically at the tumor site as opposed to release to other tissues. This is so because the concentration of the enzyme at the tumor site is higher than its concentration at other tissues due to the coating of the tumor cells with the antibody-enzyme conjugate.

ADEPT™ Technology

ADEPT™ is a novel approach to utilizing non-internalizing monoclonal antibodies that remain bound to the target cell surface. ADEPT™ involves the combination of two relatively non-toxic agents to achieve potent antitumor activity specifically within tumor tissue. In the first step, a tumor-reactive mAb is used to target catalytic enzymes to the surface of cancer cells. In the second step, inactive forms of anti-cancer drugs, e.g. prodrugs, are administered and converted to their active cytotoxic form by the enzyme that has been localized to the tumor microenvironment. This approach results in enzymatic conversion of prodrug to drug specifically in tumor tissue, thus reducing exposure of normal tissue to the drug while maximizing concentrations in tumor tissue.

Antibodies

Specific antisera are prepared by conventional techniques of immunization and collection of sera. These antisera are then absorbed with, for example, normal cells to remove those unwanted antibodies against cells other than the target cells. The remaining antibodies will be directed against predominantly the target cells of choice, for example, tumor cells. Tumor specific antibodies are for example raised against neoplastic tissue by techniques described by Ghose et al., British Medical Journal (1972) 3, 495-499.

Tumor specific antibodies against neoplasms of lymphatic and hematopoietic tissues including lymphosarcoma, chronic lymphatic leukemia, Hodgkin's disease, carcinoma of the ovary, breast and testicles, and other epithelial tissues and melanoma are contemplated.

The antibody of the immunoconjugate of the invention includes any antibody which binds specifically to a tumor-associated antigen. Examples of such antibodies include, but are not limited to, those which bind specifically to antigens found on carcinomas, melanomas, lymphomas, and bone and soft tissue sarcomas as well as other tumors. In one aspect, the antibodies remain bound to the cell surface for extended periods or are internalized very slowly. These antibodies may be polyclonal or preferably, monoclonal, may be intact antibody molecules or fragments containing the active binding region of the antibody, e.g., Fab or F(ab′)₂, and can be produced using techniques well established in the art. See, e.g., R. A. DeWeger et al., Immunological Rev., 62: 29-45, 1982 (tumor-specific polyclonal antibodies produced and used in conjugates): Yeh et al., Proc. Natl. Acad. Sci. U.S.A., 76:2927, 1979; Brown et al., J. Immun., 127:539, 1981 (tumor-specific monoclonal antibodies produced); and Mach et al., in Monoclonal Antibodies for Cancer Detection and Therapy, R. W. Baldwin et al., eds., pp 53-64, Academic Press, 1985 (antibody fragments produced and used to localize tumor cells). In addition, if monoclonal antibodies are used, the antibodies may be of mouse or human origin or chimeric antibodies (see, e.g., Oi, Biotechniques, 4:214, 1986).

Examples of antibodies which may be used to deliver the beta-lactamase to the tumor site include, but are not limited to, L6, an IgG2a monoclonal antibody (hybridoma deposit no. ATCC HB8677) that binds to a glycoprotein antigen on human lung carcinoma cells (Hellstrom et al., Proc. Natl. Acad. Sci. U.S.A., 83:7059, 1986); 96.5, an IgG2a monoclonal antibody that is specific for p97, a melanoma-associated antigen (Brown, et al., J. Immunol. 127:539, 1981); 1F5, an IgG2a monoclonal antibody (hybridoma deposit no. ATCC HB9645) that is specific for the CD-20 antigen on normal and neoplastic B cells (Clark et al., Proc. Natl. Acad. Sci. U.S.A., 82:1766, 1985), and L49 antibody, also specific for p97. The L49 antibody component binds to the p97 cell surface antigen, which is non-internalizing and highly expressed on melanoma, as well as some ovarian, breast and lung carcinomas. More specifically, the immunoconjugate component may be an L49 single chain Fv fused to beta-lactamase as described in McDonagh et al., Bioconjugate Chem. 14:860-869, 2003, incorporated herein in its entirety by reference.

Other antibodies which may be used to deliver the enzyme to the target cell population include known antibodies for the treatment or prevention of cancer. Antibodies immunospecific for a cancer cell antigen can be obtained commercially or produced by any method known to one of skill in the art such as, e.g., recombinant expression techniques. The nucleotide sequence encoding antibodies immunospecific for a cancer cell antigen can be obtained, e.g., from the GenBank database or a database like it, the literature publications, or by routine cloning and sequencing. Examples of antibodies available for the treatment of cancer include, but are not limited to, humanized anti-HER2 monoclonal antibody, HERCEPTIN® (trastuzumab; Genentech) for the treatment of patients with metastatic breast cancer; RITUXAN® (rituximab; Genentech) which is a chimeric anti-CD20 monoclonal antibody for the treatment of patients with non-Hodgkin's lymphoma; OvaRex (AltaRex Corporation, MA) which is a murine antibody for the treatment of ovarian cancer; Panorex (Glaxo Wellcome, NC) which is a murine IgG₂ₐ antibody for the treatment of colorectal cancer; Cetuximab Erbitux (Imclone Systems Inc., NY) which is an anti-EGFR IgG chimeric antibody for the treatment of epidermal growth factor positive cancers, such as head and neck cancer; Vitaxin (MedImmune, Inc., MD) which is a humanized antibody for the treatment of sarcoma; Campath I/H (Leukosite, MA) which is a humanized IgG₁ antibody for the treatment of chronic lymphocytic leukemia (CLL); Smart MI95 (Protein Design Labs, Inc., CA) which is a humanized anti-CD33 IgG antibody for the treatment of acute myeloid leukemia (AML); LymphoCide (Immunomedics, Inc., NJ) which is a humanized anti-CD22 IgG antibody for the treatment of non-Hodgkin's lymphoma; Smart ID10 (Protein Design Labs, Inc., CA) which is a humanized anti-HLA-DR antibody for the treatment of non-Hodgkin's lymphoma; Oncolym (Techniclone, Inc., CA) which is a radiolabeled murine anti-HLA-Dr10 antibody for the treatment of non-Hodgkin's lymphoma; Allomune (BioTransplant, CA) which is a humanized anti-CD2 mAb for the treatment of Hodgkin's Disease or non-Hodgkin's lymphoma; Avastin (Genentech, Inc., CA) which is an anti-VEGF humanized antibody for the treatment of lung and colorectal cancers; Epratuzamab (Immunomedics, Inc., NJ and Amgen, CA) which is an anti-CD22 antibody for the treatment of non-Hodgkin's lymphoma; and CEAcide (Immunomedics, NJ) which is a humanized anti-CEA antibody for the treatment of colorectal cancer.

Other antibodies useful in the treatment of cancer include, but are not limited to, antibodies against the following antigens: CA125 (ovarian), CA15-3 (carcinomas), CA19-9 (carcinomas), L6 (carcinomas), Lewis Y (carcinomas), Lewis X (carcinomas), alpha fetoprotein (carcinomas), CA 242 (colorectal), placental alkaline phosphatase (carcinomas), prostate specific antigen (prostate), prostatic acid phosphatase (prostate), epidermal growth factor (carcinomas), MAGE-1 (carcinomas), MAGE-2 (carcinomas), MAGE-3 (carcinomas), MAGE-4 (carcinomas), anti-transferrin receptor (carcinomas), p97 (melanoma), MUC1-KLH (breast cancer), CEA (colorectal), gp100 (melanoma), MART1 (melanoma), PSA (prostate), IL-2 receptor (T-cell leukemia and lymphomas), CD20 (non-Hodgkin's lymphoma), CD52 (leukemia), CD33 (leukemia), CD22 (lymphoma), human chorionic gonadotropin (carcinoma), CD38 (multiple myeloma), CD40 (lymphoma), mucin (carcinomas), P21 (carcinomas), MPG (melanoma), and Neu oncogene product (carcinomas). Some specific, useful antibodies include, but are not limited to, BR96 mAb (Trail, P. A., Willner, D., Lasch, S. J., Henderson, A. J., Hofstead, S. J., Casazza, A. M., Firestone, R. A., Hellström, I., Hellström, K. E., “Cure of Xenografted Human Carcinomas by BR96-Doxorubicin Immunoconjugates” Science 1993, 261, 212-215), BR64 (Trail, P A, Willner, D, Knipe, J., Henderson, A. J., Lasch, S. J., Zoeckler, M. E., Trailsmith, M. D., Doyle, T. W., King, H. D., Casazza, A. M., Braslawsky, G. R., Brown, J. P., Hofstead, S. J., (Greenfield, R. S., Firestone, R. A., Mosure, K., Kadow, D. F., Yang, M. B., Hellstrom, K. E., and Hellstrom, I. “Effect of Linker Variation on the Stability, Potency, and Efficacy of Carcinoma-reactive BR64-Doxorubicin Immunoconjugates” Cancer Research 1997, 57, 100-105, mAbs against the CD40 antigen, such as S2C6 mAb (Francisco, J. A., Donaldson, K. L., Chace, D., Siegall, C. B., and Wahl, A. F. “Agonistic properties and in vivo antitumor activity of the anti-CD-40 antibody, SGN-14” Cancer Res. 2000, 60, 3225-3231), mAbs against the CD70 antigen, such as 1F6 mAb and 2F2 mAb, and mAbs against the CD30 antigen, such as AC10 (Bowen, M. A., Olsen, K. J., Cheng, L., Avila, D., and Podack, E. R. “Functional effects of CD30 on a large granular lymphoma cell line YT” J. Immunol., 151, 5896-5906, 1993: Wahl et al., 2002 Cancer Res. 62(13):3736-42). Many other internalizing antibodies that bind to tumor associated antigens can be used and have been reviewed (Franke, A. E., Sievers, E. L., and Scheinberg, D. A., “Cell surface receptor-targeted therapy of acute myeloid leukemia: a review” Cancer Biother Radiopharm. 2000, 15, 459-76; Murray, J. L., “Monoclonal antibody treatment of solid tumors: a coming of age” Semin Oncol. 2000, 27, 64-70; Breitling, F., and Dubel, S., Recombinant Antibodies, John Wiley, and Sons, New York, 1998).

In another specific embodiment, other antibodies which may be used to deliver the enzyme to the target cell population include known antibodies for the treatment or prevention of an autoimmune disease. Antibodies immunospecific for an antigen of a cell that is responsible for producing autoimmune antibodies can be obtained from any organization (e.g., a university scientist or a company) or produced by any method known to one of skill in the art such as, e.g., chemical synthesis or recombinant expression techniques. In another embodiment, useful antibodies are immunospecific for the treatment of autoimmune diseases include, but are not limited to, Anti-Nuclear Antibody; Anti-ds DNA; Anti-ss DNA, Anti-Cardiolipin Antibody IgM, IgG; Anti-Phospholipid Antibody IgM, IgG; Anti-SM Antibody; Anti-Mitochondrial Antibody; Thyroid Antibody; Microsomal Antibody; Thyroglobulin Antibody; Anti-SCL-70; Anti-Jo; Anti-U₁RNP; Anti-La/SSB; Anti SSA; Anti-SSB; Anti-Perital Cells Antibody; Anti-Histones; Anti-RNP; C-ANCA; P-ANCA; Anti centromere; Anti-Fibrillarin, and Anti-GBM Antibody.

In certain embodiments, useful antibodies can bind to both a receptor or a receptor complex expressed on an activated lymphocyte. The receptor or receptor complex can comprise an immunoglobulin gene superfamily member, a TNF receptor superfamily member, an integrin, a cytokine receptor, a chemokine receptor, a major histocompatibility protein, a lectin, or a complement control protein. Non-limiting examples of suitable immunoglobulin superfamily members are CD2, CD3, CD4, CD8, CD19, CD22, CD28, CD79, CD90, CD152/CTLA-4, PD-1, and ICOS. Non-limiting examples of suitable TNF receptor superfamily members are CD27, CD40, CD95/Fas, CD134/OX40, CD137/4-1BB, TNF-R1, TNFR-2, RANK, TACI, BCMA, osteoprotegerin, Apo2/TRAIL-R1, TRAIL-R2, TRAIL-R3, TRAIL-R4, and APO-3. Non-limiting examples of suitable integrins are CD11a, CD11b, CD11c, CD18, CD29, CD41, CD49a, CD49b, CD49c, CD49d, CD49e, CD49f, CD103, and CD104. Non-limiting examples of suitable lectins are C-type, S-type, and I-type lectin.

In another specific embodiment, other antibodies which may be used to deliver the enzyme to the target cell population include those immunospecific for a viral or a microbial antigen. The antibodies may be chimeric, humanized or human monoclonal antibodies. As used herein, the term “viral antigen” includes, but is not limited to, any viral peptide, polypeptide protein (e.g., HIV gp120, HIV nef, RSV F glycoprotein, influenza virus neuraminidase, influenza virus hemagglutinin, HTLV tax, herpes simplex virus glycoprotein (e.g., gB, gC, gD, and gE) and hepatitis B surface antigen) that is capable of eliciting an immune response. As used herein, the term “microbial antigen” includes, but is not limited to, any microbial peptide, polypeptide, protein, saccharide, polysaccharide, or lipid molecule (e.g., a bacterial, fungi, pathogenic protozoa, or yeast polypeptide including, e.g., LPS and capsular polysaccharide 5/8) that is capable of eliciting an immune response.

Antibodies immunospecific for a viral or microbial antigen can be obtained commercially, for example, from BD Biosciences (San Francisco, Calif.), Chemicon International, Inc. (Temecula, Calif.), or Vector Laboratories, Inc. (Burlingame, Calif.) or produced by any method known to one of skill in the art such as, e.g., chemical synthesis or recombinant expression techniques. The nucleotide sequence encoding antibodies that are immunospecific for a viral or microbial antigen can be obtained, e.g., from the GenBank database or a database like it, literature publications, or by routine cloning and sequencing.

Examples of antibodies available useful for the treatment of viral infection or microbial infection include, but are not limited to, SYNAGIS (MedImmune, Inc., MD) which is a humanized anti-respiratory syncytial virus (RSV) monoclonal antibody useful for the treatment of patients with RSV infection; PRO542 (Progenics) which is a CD4 fusion antibody useful for the treatment of HIV infection; OSTAVIR (Protein Design Labs, Inc., CA) which is a human antibody useful for the treatment of hepatitis B virus; PROTOVIR (Protein Design Labs, Inc., CA) which is a humanized IgG₁ antibody useful for the treatment of cytomegalovirus (CMV); and anti-LPS antibodies.

Other antibodies useful for treatment of viral disease include, but are not limited to, antibodies against antigens of pathogenic viruses, including as examples and not by limitation: Poxviridae, Herpesviridae, Herpes Simplex virus 1, Herpes Simplex virus 2, Adenoviridae, Papovaviridae, Enteroviridae, Picomaviridae, Parvoviridae, Reoviridae, Retroviridae, influenza viruses, parainfluenza viruses, mumps, measles, respiratory syncytial virus, rubella, Arboviridae, Rhabdoviridae, Arenaviridae, Hepatitis A virus, Hepatitis B virus, Hepatitis C virus, Hepatitis E virus, Non-A/Non-B Hepatitis virus, Rhinoviridae, Coronaviridae, Rotoviridae, and Human Immunodeficiency Virus.

Other antibodies which may be used to deliver the enzyme to the target cell population useful in the treatment of infectious diseases include, but are not limited to, antibodies against the antigens from pathogenic strains of bacteria (Streptococcus pyogenes, Streptococcus pneumoniae, Neisseria gonorrheae, Neisseria meningitidis, Corynebacterium diphtheriae, Clostridium botulinum, Clostridium perfringens, Clostridium tetani, Hemophilus influenzae, Klebsiella pneumoniae, Klebsiella ozaenas, Klebsiella rhinoscleromotis, Staphylococc aureus, Vibrio colerae, Escherichia coli, Pseudomonas aeruginosa, Campylobacter (Vibrio) fetus, Aeromonas hydrophila, Bacillus cereus, Edwardsiella tarda, Yersinia enterocolitica, Yersinia pestis, Yersinia pseudotuberculosis, Shigella dysenteriae, Shigella flexneri, Shigella sonnei, Salmonella typhimurium, Treponema pallidum, Treponema pertenue, Treponema carateneum, Borrelia vincentii, Borrelia burgdorferi, Leptospira icterohemorrhagiae, Mycobacterium tuberculosis, Pneumocystis carinii, Francisella tularensis, Brucella abortus, Brucella suis, Brucella melitensis, Mycoplasma spp., Rickettsia prowazeki, Rickettsia tsutsugumushi, Chlamydia spp.); pathogenic fungi (Coccidioides immitis, Aspergillus fumigatus, Candida albicans, Blastomyces dermatitidis, Cryptococcus neoformans, Histoplasma capsulatum); protozoa (Entomoeba histolytica, Toxoplasma gondii, Trichomonas tenas, Trichomonas hominis, Trichomonas vaginalis, Tryoanosoma gambiense, Trypanosoma rhodesiense, Trypanosoma cruzi, Leishmania donovani, Leishmania tropica, Leishmania braziliensis, Pneumocystis pneumonia, Plasmodium vivax, Plasmodium falciparum, Plasmodium malaria); or Helminiths (Enterobius vermicularis, Trichuris trichiura, Ascaris lumbricoides, Trichinella spiralis, Strongyloides stercoralis, Schistosoma japonicum, Schistosoma mansoni, Schistosoma haematobium, and hookworms).

An alternative strategy is to use antibodies that internalize, providing that the prodrug can also internalize, or that a sufficient amount of antibody also remains on the surface of the cell. An example of such antibodies may be found in Cancer Research 56:2183 (1990).

Enzyme Component

In one aspect, the enzyme component of the immunoconjugate includes any enzyme capable of hydrolyzing the CO—N bond of a beta-lactam. These enzymes are available commercially, such as E. coli or B. cereus beta-lactamases, from Sigma-Aldrich Corp., St. Louis, Mo., USA. These and other beta-lactamases may be cloned and expressed using recombinant DNA techniques well known in the art.

The beta-lactamases of this invention can be covalently bound to antibodies by techniques well known in the art such as the use of the heterobifunctional crosslinking reagents SPDP (N-succinimidyl-3-(2-pyridyldithio)propionate) or SMCC (succinimidyl 4-(N-maleimidomethyl)cyclohexane-1-carboxylate (see, e.g., Thorpe et al., Immunol. Rev., 62: 119, 1982; Lambert et al., supra, at p. 12038; Rowland et al., supra, at pp 183-184; Gallego et al., supra, at pp. 737-7138). Alternatively, fusion proteins comprising at least the antigen binding region of an antibody linked to at least a functionally active portion of a beta-lactamase can be constructed using recombinant DNA techniques well known in the art (see, e.g., Neuberger et al., Nature, 312:604, 1984). These fusion proteins act in essentially the same manner as the antibody-enzyme conjugates described herein.

Compositions

A pharmaceutical or veterinary composition or medicament (hereinafter simply referred to as a pharmaceutical composition) is provided containing a novel melphalan prodrug, antibody-enzyme conjugate or antibody-prodrug conjugate as described herein is provided for administration to a patient having a disease (e.g. a tumor) susceptible to treatment. The pharmaceutical composition is administered in an amount sufficient to cure, or at least partially arrest, the symptoms of the disease and its complications. An amount adequate to accomplish this is defined as a therapeutically- or pharmaceutically-effective dose. In therapeutic regimes, the pharmaceutical composition is usually administered in several dosages until a sufficient response has been achieved. The pharmaceutical composition can include a pharmaceutically acceptable carrier, excipient, or diluent.

The pharmaceutical compositions may be in any form that allows for the composition to be administered to an animal subject. For example, the composition may be in the form of a solid, liquid or gas (e.g., aerosol, vapor, nebulized). Typical routes of administration include, without limitation, oral, topical, parenteral, sublingual, rectal, vaginal, ocular, and intranasal. The term parenteral as used herein includes subcutaneous injections, intravenous, intramuscular, intrastemal injection or infusion techniques. Pharmaceutical compositions of are formulated so as to allow the active ingredients contained therein to be bioavailable upon administration of the composition to an animal subject. Compositions that will be administered to a subject take the form of one or more dosage units, where for example, a tablet may be a single dosage unit, and a container of a compound of the invention in aerosol form may hold a plurality of dosage units.

Materials used in preparing the pharmaceutical compositions should be pharmaceutically pure and non-toxic in the amounts used. It will be evident to those of ordinary skill in the art that the optimal dosage of the active ingredient(s) in the pharmaceutical composition will depend on a variety of factors. Relevant factors include, without limitation, the type of subject (e.g., human), the particular form of the active ingredient, the manner of administration, and the composition employed.

In general, the pharmaceutical composition includes an (where “a” and “an” refers here, and throughout this specification, as one or more) active compounds of the invention in admixture with one or more carriers. The carrier(s) may be particulate, so that the compositions are, for example, in tablet or powder form. The carrier(s) may be liquid, with the compositions being, for example, an oral syrup or injectable liquid. In addition, the carrier(s) may be gaseous, so as to provide an aerosol composition useful in, e.g., inhalatory administration.

When intended for oral administration, the composition is preferably in either solid or liquid form, where semi-solid, semi-liquid, suspension and gel forms are included within the forms considered herein as either solid or liquid.

As a solid composition for oral administration, the composition may be formulated into a powder, granule, compressed tablet, pill, capsule, chewing gum, wafer or the like form. Such a solid composition will typically contain one or more inert diluents or edible carriers. In addition, one or more of the following adjuvants may be present: binders such as carboxymethylcellulose, ethyl cellulose, microcrystalline cellulose, or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin, a flavoring agent such as peppermint, methyl salicylate or orange flavoring, and a coloring agent.

When the composition is in the form of a capsule, e.g., a gelatin capsule, it may contain, in addition to materials of the above type, a liquid carrier such as polyethylene glycol, cyclodextrin, or a fatty oil.

The composition may be in the form of a liquid, e.g., an elixir, syrup, solution, emulsion, or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition for administration by injection, one or more of a surfactant, preservative, wetting agent, dispersing agent, suspending agent, buffer, stabilizer, and isotonic agent may be included.

The liquid pharmaceutical compositions, whether they are solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. In one aspect, physiological saline is the adjuvant. In another aspect, an injectable pharmaceutical composition is sterile.

A liquid composition intended for either parenteral or oral administration should contain an amount of a compound of a melphalan prodrug such that a suitable dosage will be obtained. Typically, this amount is at least 0.01% of a compound of the invention in the composition however the precise dose will depend in large part on the drug selected for incorporation. When intended for oral administration, this amount may be varied to be between 0.1% and about 80% of the weight of the composition. Preferred oral compositions contain between about 4% and about 50% of the compound. In one aspect, compositions and preparations according to the present invention are prepared so that a parenteral dosage unit contains between 0.01% to 2% by weight of active compound.

The pharmaceutical composition may be intended for topical administration, in which case the carrier may suitably comprise a solution, emulsion, ointment or gel base. The base, for example, may comprise one or more of the following: petrolatum, lanolin, polyethylene glycols, beeswax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of a compound of the present invention of from about 0.1% to about 10% w/v (weight per unit volume).

The composition may be intended for rectal administration, in the form, e.g., of a suppository which will melt in the rectum and release the drug. The composition for rectal administration may contain an oleaginous base as a suitable nonirritating excipient. Such bases include, without limitation, lanolin, cocoa butter and polyethylene glycol.

The composition may include various materials that modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition may consist of gaseous dosage units, e.g., it may be in the form of an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi-phasic, or tri-phasic systems in order to deliver the active ingredient(s). Delivery of the aerosol includes the necessary container, activators, valves, subcontainers, spacers and the like, which together may form a kit. In one aspect, aerosols may be determined by one skilled in the art, without undue experimentation.

Whether in solid, liquid or gaseous form, the pharmaceutical composition may contain one or more known pharmacological agents used in the treatment of cancer.

The pharmaceutical compositions may be prepared by methodology well known in the pharmaceutical art. For example, a composition intended to be administered by injection can be prepared by combining a prodrug compound with water so as to form a solution. A surfactant may be added to facilitate the formation of a homogeneous solution or suspension. Surfactants include compounds that non-covalently interact with a prodrug compound so as to facilitate dissolution or homogeneous suspension of the active compound in the aqueous delivery system.

Synthesis

The nitrogen mustard compounds may have the following general formula: 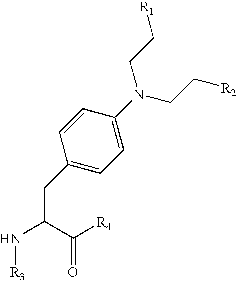
where R₁, R₂=independently halogens, O-mesylate, or O-tosylate,
R₃=H or lower alkyl groups C₁-C₆,
R₄=OH, PEG, alkoxy, cycloalkoxy, aryloxy, arylalkoxy, —NH₂, NHR, NRR′, where R and R′ are, independently, alkyl, alkenyl, alkynyl, heteroaryl alkyl, substituted alkyl, substituted aryl, substituted arylalkyl, heteroaryl, or is comprised of a peptide as follows:

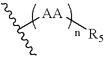

where AA is any given amino acid,

n=1 to 12 and
R₅ represents the manner in which the C-terminal amino acid or C-terminal amino acid derivative is capped at the carboxy terminus, if at all, and a pharmaceutically acceptable salt or solvent thereof. The amino acids include but are not limited to natural or synthetic, D or L, R or S, essential or non essential, and alpha amino acids. R₅ may range from a wide variety of amino acids, including but certainly are not limited to: alanine (including □-alanine and N-methylalanine), asparagine (including □-asparagine and N-methyl asparagine), aspartic acid, cysteine, glutamic acid, phenylalanine, glycine, histidine, isoleucine, lysine, leucine, methionine, asparagines, proline, glutamine, arginine, serine, threonine, valine, tryptophan, tyrosine, glutamic acid, citrulline, 4-aminobutanoic acid, 5-aminopentanoic acid, and 6-aminohexanoic acid.

In one aspect, the nitrogen mustard compound is melphlan and derivatives thereof.

In one embodiment, the prodrugs include the nitrogen mustard compounds as a drug unit D. The drug unit may be linked to an enzyme substrate as shown in the general formula below: 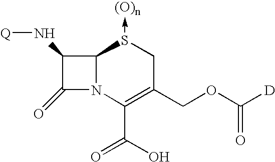
and pharmaceutically acceptable salts and solvates thereof, wherein
Q=H or salt thereof, C═O-alkyl, C═O-PEG, C═O-cycloalkyl, C═O-aryl, C═O-arylalkyl, CO₂R, or CONRR′ where R and R′ are, independently, an alkyl, alkenyl, alkynyl, heteroaryl alkyl, substituted alkyl, substituted aryl, substituted arylalkyl, heteroaryl, PEG, cycloalkyl, aryl, or arylalkyl; and n=0, 1, or 2.

More specifically Q could be the following:

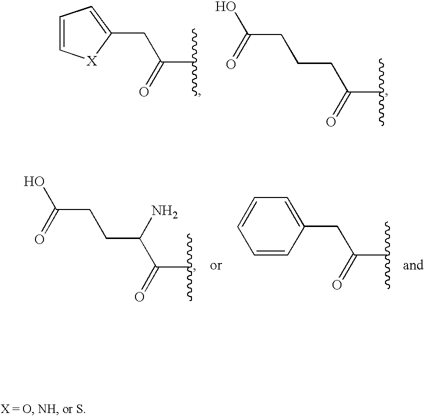

Cephalosporin and sulfoxides of cephalosporin are excellent substrates for beta-lactamase enzyme. The sulfoxide of cephalosporin has been shown to be a better substrate than the parent cephalosporin. Louis N. Jungheim, Timothy A. Shepherd, and Damon L. Meyer. J. Org. Chem. 57: 2334-40, 1992. Prodrugs which are rapidly hydrolyzed by the beta-lactamase, and which exhibit high affinity for the enzyme, are expected to offer a therapeutic advantage in an ADEPT™ delivery system. The sulfoxide has also been shown to exhibit superior solution stability. Louis N. Jungheim, Timothy A. Shepherd, and Damon L. Meyer. J. Org. Chem. 57: 2334-40, 1992. The presence of the sulfoxide moiety precludes double bond migration to the undesired Δ-2 cephem olefin isomer.

Ester Synthesis. Ester stability in plasma varies with steric bulk for many esters. A series of esters were synthesized that varied in bulk, stereochemistry, and electronics and were evaluated in vitro. Melphalan 2 served as the starting point for the synthesis of the drugs. Most of the melphalan esters shown in Table 1 were routinely prepared with melphalan and the corresponding alcohol in a Dean-Stark apparatus or by simply refluxing in toluene or benzene in the presence of molecular sieves. In the case of the t-butyl ester (Scheme 1, 21), isobutylene chemistry was employed. Purification for all esters was accomplished by silica gel chromatography to afford the free amine or by reversed-phase HPLC with 0.1% TFA or 0.1% formic acid to give the desired ammonium salt.

TABLE 1
ESTER SYNTHESIS

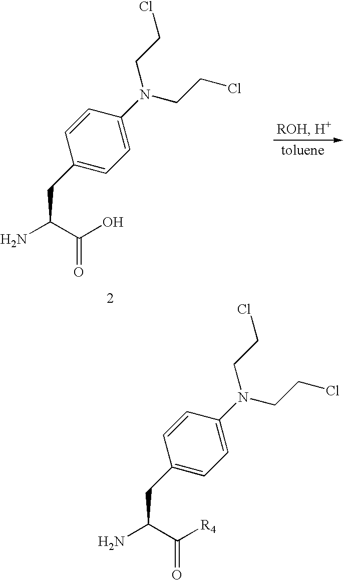

| Product No. | R₄ |
|---|---|
| 3 | 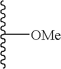 |
| 4 | 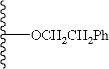 |
| 5 | 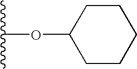 |
| 6 | 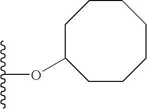 |
| 7 | 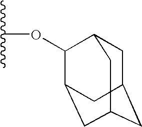 |
| 8 | 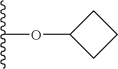 |
| 9 | 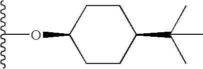 |
| 10 | 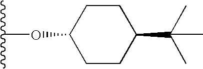 |
| 11 | 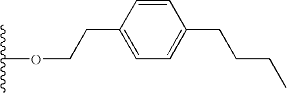 |
| 12 | 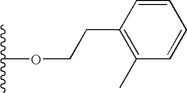 |
| 13 | 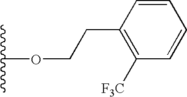 |
| 14 | 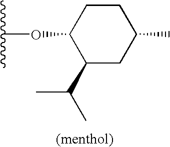 |
| 15 | 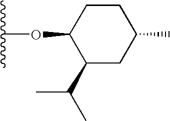 |

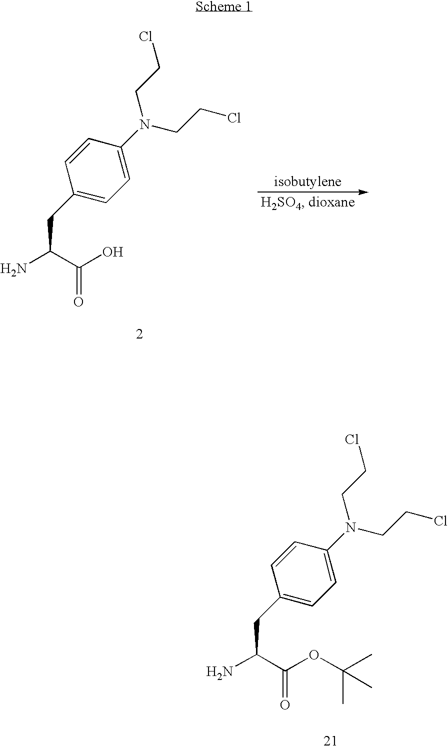

Peptide Synthesis. The melphalan peptides were carried out in a straightforward manner (Table 2). Boc-melphalan was easily prepared followed by peptide coupling with the carboxyl-modified/protected amino acid using the DEPC coupling reagent in methylene chloride. Lastly, Boc-removal with TFA followed by purification led to the appropriate melphalan dipeptides.

For peptide derivatives of chlorambucil (CA), an amide of CA can be synthesized with, for example, a lysine residue that contains 2 amines. One of the lysine amines could be attached to the CA and the other to the cephalosporin thereby hooking the CA to the cephalosporin with a lysine in-between. Ester derivatives of CA are also envisioned.

Commercially available 7-aminocephalosporanic acid (7-ACA) was the starting point for the preparation of the glutaryl-cephalosporin derivatives. In a one-pot fashion, 7-ACA (Scheme 2, 22) was saponified to form the alcohol, treated with glutaric anhydride, and bis-ester protected with freshly prepared diphenyldiazomethane to afford 23. It was apparent that one of the carboxyl groups preferentially reacted with diphenyldiazomethane according to HPLC analysis. Mono-esterification proceeded to near completion after 1 h while the bis-diphenylmethyl ester required at least 24 h. The product was resistant to precipitation unlike the 7-phenylacetamido derivative and, due to its instability on normal phase silica gel, purification via reverse-phase HPLC under neutral conditions was chosen. The resulting purified alcohol 23 was converted to the activated carbonate and the sulfide was oxidized to give the key intermediate 24.

TABLE 2
PEPTIDE SYNTHESIS

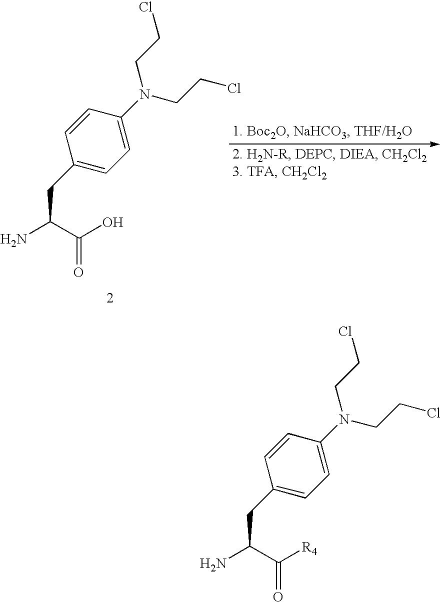

| Product No. | R₄ |
|---|---|
| 16 | 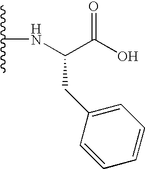 |
| 17 | 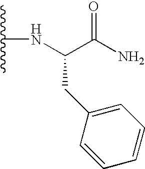 |
| 18 | 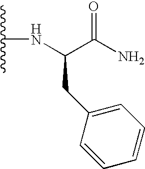 |
| 19 | 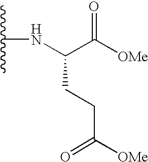 |
| 20 | 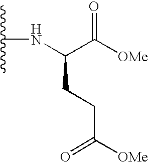 |

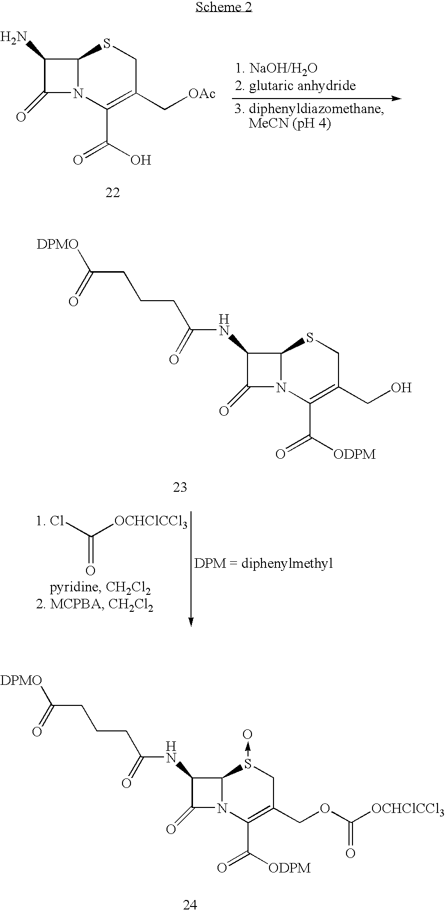

Cephalosporin coupling. As disclosed in Table 3, the desired cephalosporin was coupled to the melphalan derivative of interest in THF (Scheme 3) followed by deprotection with excess TFA and anisole to afford the prodrug. In the case of the melphalan t-butyl ester prodrugs 33 and 34, a reaction mixture of 1% TFA and careful monitoring by BPLC afforded selective benzhydryl ester cleavage in high overall yield. All prodrugs were purified by reversed-phase HPLC using MeCN-water containing 0.1% formic acid or 0.1% trifluoroacetic acid followed by lyophilization.

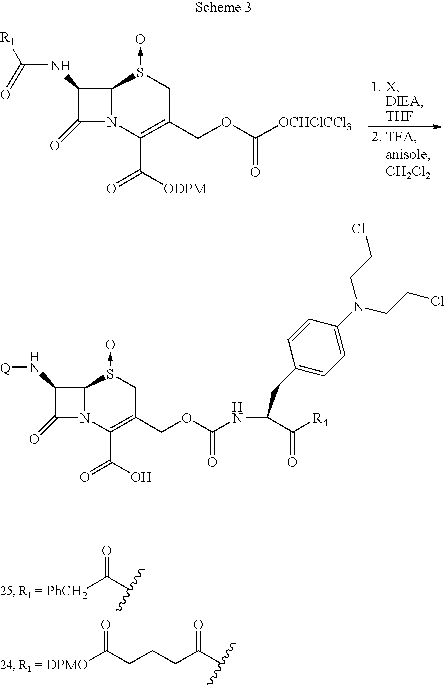

TABLE 3

| | Product/ | | | |
|---|---|---|---|---|
| X (Cmpd #) | Formula # | R₁ | R₄ | Q |
| 2 | 26 | 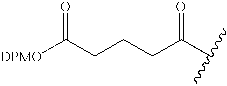 | 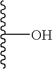 | 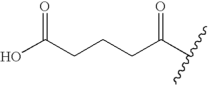 |
| 3 | 27 | 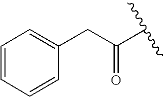 | 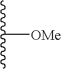 | 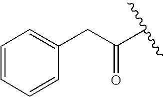 |
| 4 | 28 | 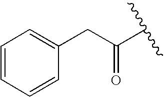 | 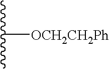 | 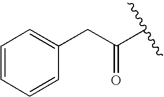 |
| 5 | 29 | 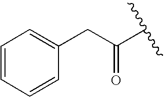 | 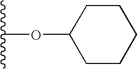 | 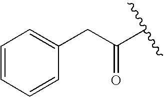 |
| 5 | 30 | 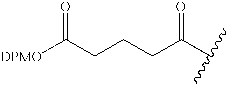 | 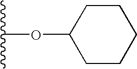 | 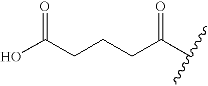 |
| 8 | 31 | 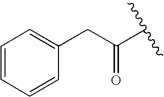 | 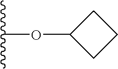 | 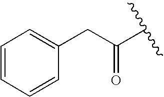 |
| 8 | 32 | 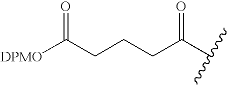 | 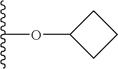 | 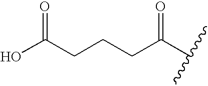 |
| 21 | 33 | 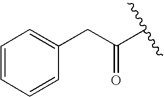 | 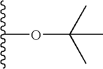 | 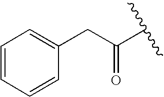 |
| 21 | 34 | 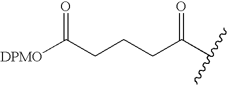 | 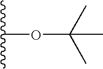 | 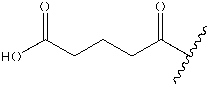 |
| 17 | 35 | 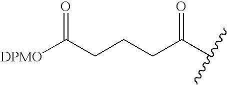 | 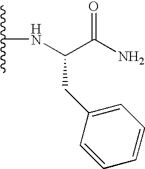 | 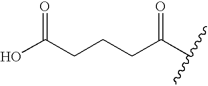 |
| 18 | 36 | 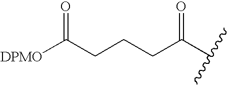 | 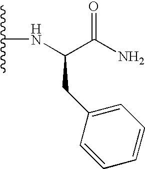 | 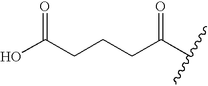 |

Melphalan Methyl Ester (3)

Melphalan (197 mg, 645 mmol) was suspended in methanol (20 mL), followed by the addition of concentrated sulfuric acid (ca. 0.5 mL). No melphalan was detected after 16 h of refluxing. The reaction mixture was concentrated, taken up in EtOAc (100 mL) and washed successively with sodium bicarbonate (50 mL×2), water (50 mL), and brine. The organic layer was dried (MgSO₄), concentrated, and purified by reversed phase prep-HPLC (C₁₈ column) using a MeCN-water gradient with 0.1% TFA. Yield: 95 mg (46%). R_{f} 0.35 (100% EtOAc); ES-MS m/z 319.17 [M+H]⁺; UV δₘₐₓ 215, 265, 300 nm.

Melphalan Cyclobutyl Ester (8)—General Procedure A

Melphalan 2 (250 mg, 0.78 mmol) and cyclobutanol (1.0 g, 13.7 mmol, ca. 20 eq.) were suspended in toluene (5 mL, 0.15 M) in the presence of 3 Å molecular sieves. To this was addedp-toluenesulfonic acid (0.44 g, 23.3 mmol, 3.0 eq.). The mixture was heated to reflux and monitored. By HPLC analysis, the reaction was complete in 2 h. The contents were cooled, filtered and washed with methylene chloride. The filtrate was evaporated to an oil that was subjected to purification by reverse-phase HPLC (C₁₈ column, 0.1% TFA in a water-MeCN gradient). The product was isolated as an off-white solid. Yield: 210 mg (57%). R_{f} 0.40 (100% EtOAc); ES-MS m/z 359.2 [M+H]⁺; UV δₘₐₓ 215, 265, 300 mn.

Melphalan Phenethyl Ester (4)

Melphalan (0.4 g, 1.2 mmol), phenethyl alcohol (1 mL, 8.35 mmol, 6.4 eq.), p-toluenesulfonic acid (0.55 g, 2.88 mmol, 2.2 eq.) and toluene (5 mL, 0.2 M) were used according to the general procedure A. The reaction was purified by reverse-phase HPLC (C₁₈ column, 0.1% TFA in a water-MeCN gradient). The product 13 was isolated as an off-white solid. Yield: 0.4 g (58%). ES-MS m/z 409.3 [M+H]⁺; UV δₘₐₓ 215, 265, 300 nm.

Melphalan Cyclohexyl Ester (5)

Melphalan (0.905 g, 2.96 mmol), cyclohexanol (2 mL, 19.1 mmol, ca. 6.5 eq.), p-toluenesulfonic acid (1.24 g, 6.52 mmol, 2.2 eq.) and dry toluene (15 mL, 0.2 M) were used according to the general procedure A. The reaction was purified over silica gel eluting with 100% ethyl acetate. The product was isolated as a light yellow oil. The easily handled trifluoroacetate salt may be obtained by purifying by reversed phase prep-HPLC (C₁₈ column) using a MeCN-water gradient with 0.1% TFA. Yield: 0.51 g (45%). R_{f} 0.40 (100% EtOAc); ES-MS m/z 387.42 [M+H]⁺; UV δₘₐₓ 215, 265, 300 nm. ¹H NMR (DMSO-d₆) δ 8.37 (3H, s, NH₃), 7.03 (1H, d, J=8.8 Hz, Ar—CH×2), 6.70 (2H, d, J=8.8 Hz, Ar—CH×2), 4.65-4.73 (1H, m, cyclohexyl-CH), 4.16 (1H, dd, J_{CH,B}=7.2 Hz, J_{CH,A}=6.6 Hz, mel-CH), 3.64-3.75 (8H, m, NCH₂CH₂Cl×2), 3.03 (1H, dd, J_{CH,A}=6.0 Hz, J_{A,B}=14.0 Hz, mel-CH_{2A}), 3.00 (1H, dd, J_{CH,B}=7.6 Hz, J_{A,B}=13.8 Hz, mel-CH_{2B}), 1.12-1.76 (10H, m, cyclohexyl).

Melphalan Cyclooctyl Ester (6)

Melphalan (0.4 g, 1.2 mmol), cyclooctanol (0.52 mL, 3.93 mmol, 3 eq.), p-toluenesulfonic acid (0.55 g, 2.88 mmol, 2.2 eq.) and toluene (5 mL, 0.2 M) were used according to the general procedure A. The reaction was purified by reverse-phase HPLC (C₁₈ column, 0.1% TFA in a water-MeCN gradient). The product was isolated as an off-white solid. Yield: 220 mg (32%). ES-MS m/z 415.3 [M+H]⁺; UV δₘₐₓ 215, 265, 300 nm.

Melphalan 2-adamantanyl ester (7)

Melphalan (400 mg, 1.31 mmol), 2-adamantanol (1.0 g, 6.5 mmol, ca. 5 eq.), p-toluenesulfonic acid (0.55 g, 2.9 mmol, 2.2 eq.) and toluene (15 mL, 0.1 M) were used according to the general procedure A. The product was purified by reverse-phase HPLC (C₁₈ column, 0.1% TFA in a water-MeCN gradient). The product was isolated as an off-white solid. Yield: 160 mg (28%). R_{f} 0.20 (4:1 hexanes-acetone); ES-MS m/z 439.26 [M+H]⁺; UV δₘₐₓ 260, 300 nm.

Melphalan (4-cis-t-butyl) cyclohexyl ester (9)

Melphalan (66 mg, 0.22 mmol), (4-cis-t-butyl)cyclohexanol (0.34 g, 2.18 mmol, ca. 10 eq.), p-toluenesulfonic acid (0.12 g, 0.65 mmol, 3.0 eq.) and toluene (2 mL, 0.1 M) were used according to the general procedure A. The product was purified by reverse-phase HPLC (C₁₈ column, 0.1% TFA in a water-MeCN gradient). The product was isolated as an off-white solid. Yield: 22 mg (19%). ES-MS m/z 443.3 [M+H]⁺; UV δₘₐₓ 215, 265, 300 nm.

Melphalan (4-trans-t-butyl) cyclohexyl ester (9)

Melphalan (100 mg, 0.327 mmol), (4-trans-t-butyl)cyclohexanol (339 mg, 2.17 mmol, 6.6 eq.), p-toluenesulfonic acid (0.274 g, 1.44 mmol, 4.4 eq.) and toluene (10 mL, 0.1 M) were used according to the general procedure A. 1.5 mL of NMP was used to aid in the solubility. The product was purified by reverse-phase HPLC (C₁₈ column, 0.1% TFA in a water-MeCN gradient). The product was isolated as an off-white solid. ES-MS m/z 443.3 [M+H]⁺; UV δₘₐₓ 215, 265, 300 nm.

Melphalan (4-n-propyl)phenethyl ester (11)

Melphalan (100 mg, 0.33 mmol), (4-n-butyl)phenethyl alcohol (1.17 g, 6.56 mmol, ca. 20 eq.), p-toluenesulfonic acid (187 mg, 0.984 mmol, 3.0 eq.) and toluene (5 mL) were used according to the general procedure A. The reaction was purified by reverse-phase HPLC (C₁₈ column, 0.1% TFA in a water-MeCN gradient). The product was isolated as an off-white solid. Yield: 82 mg (43%). ES-MS m/z 465.3 [M+H]⁺; UV δₘₐₓ 215, 265, 300 nm.

Melphalan (2-methyl)phenethyl ester (12)

Melphalan (100 mg, 0.33 mmol), (2-methyl)phenethyl alcohol (893 mg, 6.56 mmol, ca. 20 eq.), p-toluenesulfonic acid (187 mg, 0.984 mmol, 3.0 eq.) and toluene (5 mL) were used according to the general procedure A. The reaction was purified by reverse-phase HPLC (C₁₈ column, 0.1% TFA in a water-MeCN gradient). The product was isolated as an off-white solid. Yield: 67 mg (38%). ES-MS m/z 423.3 [M+H]⁺; UV δₘₐₓ 215, 265, 300 nm.

Melphalan (2-trifluoromethyl)phenethyl ester (13)

Melphalan (0.4 g, 1.2 mmol), (2-trifluoromethyl)phenethyl alcohol (4.1 mL, 25 mmol, ca. 20 eq.), p-toluenesulfonic acid (0.7 g, 3.7 mmol, 3.0 eq.) and toluene (5 mL, 0.2 M) were used according to the general procedure A. The reaction was purified by reverse-phase HPLC (C₁₈ column, 0.1% TFA in a water-MeCN gradient). The product 13 was isolated as an off-white solid. Yield: 170 mg (23%). ES-MS m/z 477.2 [M+H]⁺; UV δₘₐₓ 215, 265 mn.

Melphalan Menthol Ester (14)

Melphalan (0.4 g, 1.3 mmol), (1R, 2S, 5R)-(−)-menthol (1.03 g, 6.55 mmol, ca. 5 eq.), p-toluenesulfonic acid (0.55 g, 2.9 mmol, 2.2 eq.) and toluene (15 mL) were used according to the general procedure A. The reaction was purified by flash column (SiO₂ column, 4:1 to 1:1 gradient of hexanes-acetone). The product 13 was isolated as an off-white solid. Yield: 145 mg (25%). ES-MS m/z 443.1 [M+H]⁺; R_{f} 0.19 (4:1 hexanes-acetone); UV δₘₐₓ 215, 265 nm.

Melphalan Neomenthyl Ester (15)

Melphalan (100 mg, 0.33 mmol), neomenthol (2.16 mL, 12.4 mmol, ca. 40 eq.), p-toluenesulfonic acid (0.52 g, 2.73 mmol, 8.9 eq.) and toluene (20 mL) were used according to the general procedure A. The reaction was purified by reverse-phase HPLC (C₁₈ column, 0.1% TFA in a water-MeCN gradient). The product was isolated as an off-white solid. Yield: 20 mg (11%). ES-MS m/z 443.3 [M+H]⁺; UV δₘₐₓ 215, 265, 300 nm.

Melphalan t-Butyl Ester (21)

Melphalan (0.5 g, 1.56 mmol) was suspended in 1,4-dioxane (10 mL). Sulfuric acid (0.13 mL, 2.3 mmol, 1.5 eq,) was added followed by condensed isobutylene (ca. 1 mL cooled to −30° C.) via cannula. The reaction was sealed and stirred vigorously for 16 h. The reaction was carefully vented and saturated sodium bicarbonate was slowly added while stirring. The mixture was extracted with methylene chloride (3×25 mL). An insoluble precipitate (unreacted melphalan) was filtered off. The organic layer was dried (MgSO₄), filtered and evaporated to give a yellow oil. Purification by flash column chromatography (SiO₂ column, 1:1 hexanes-EtOAc to 100% EtOAc gradient). Concentration of the fractions led to a sticky solid. Yield: 73 mg (13% overall). ES-MS m/z 361.27 [M+H]⁺; UV δₘₐₓ 260, 300 nm.

Boc-Melphalan

Melphalan (1.59 g, 4.95 mmol) was dissolved in a mixture of THF (20 mL) and aqueous NaHCO₃ (0.5 N, 30 mL, 14.8 mmol, 3.0 eq.). To this was added Boc₂O (1.64 g, 7.42 mmol, 1.5 eq.) while stirring. Reaction was complete in 20 min according to HPLC analysis. The reaction mixture was poured into a sep funnel containing EtOAc/1 M HCl (1:1) and the layers separated. The organic phase was further washed with brine followed by drying (MgSO₄), filtration and evaporation to a dark oil. The material was used without further purification in the next step. ES-MS m/z 405.19 [M+H]⁺; UV δₘₐₓ 260, 300 nm. ¹H NMR (CDCl₃) δ 7.07 (1H, d, J=8.4 Hz, Ar—CH×2), 6.63 (2H, d, J=8.8 Hz, Ar—CH×2), 4.93 (1H, d, J=7.6 Hz, NH), 4.50-4.57 (1H, m, mel-CH), 3.71 (4H, t, J=7.2 Hz, NCH₂×2), 3.61 (4H, t, J=7.2 Hz, CH₂Cl×2), 3.09 (1H, dd, J=5.6 Hz, J=13.8 Hz, mel-CH_{2A}), 3.00 (1H, dd, J=5.6 Hz, J=13.8 Hz, mel-CH_{2B}), 1.52 (9H, s, CH₃×3).

Mel-D-Phe-NH₂ (18)—General Procedure B

Boc-melphalan (0.35 g, 0.86 mmol) and D-phenylalanine amide (0.17 g, 0.86 mmol) were diluted in CH₂Cl₂ (10 mL). Diisopropylethylamine (0.38 mL, 2.16 mmol, 2.5 eq.) and DEPC (0.36 mL, 2.16 mmol, 2.5 eq.) were added while stirring. The product partially precipitated as the reaction progressed. After 1 h, no more starting material was detected according to HPLC analysis. The mixture was diluted with EtOAc (ca. 80 mL) and washed successively with 1 M HCl, water, saturated sodium bicarbonate, and brine. After drying the organic layer (MgSO₄), the solution was filtered and solvent removed. The residue was diluted with methylene chloride (10 mL) followed by the addition of TFA (3 mL). The reaction was complete in 1 h. Removal of solvent in vacuo and purification by flash column chromatography (SiO₂ column, gradient CH₂Cl₂-MeOH 95:5 to 80:20) led to the product as an off-white solid. Yield: 0.26 g (67% overall). ES-MS m/z 473.07 [M+Na]⁺; UV δₘₐₓ 260, 300 nm.

Mel-L-Phe-NH₂ (17)

Boc-melphalan (0.40 g, 1.0 mmol) and L-phenylalanine t-butyl ester (0.25 g, 1.0 mmol) were used to prepare the dipeptide 17 according to the general procedure C. Purification of the final reaction step by reverse-phase HPLC (C₁₂ column, gradient MeCN-water with 0.1% formic acid) led to the product as a beige solid after lyophilization. Yield: 0.30 g (61%). ES-MS m/z 452.37 [M+H]⁺; UV δₘₐₓ 215, 260, 300 nm. ¹H NMR (DMSO-d₆) δ 8.78 (1H, d, J_{NH,7}=6.8 Hz, phe-NH), 7.15-7.27 (5H, m, phe-Ar—CH×5), 7.07 (2H, d, J=8.8 Hz, mel-Ar—CH×2), 6.64 (2H, d, J=8.8 Hz, mel-Ar—CH×2), 4.41-4.49 (1H, m, mel-CH), 3.62-3.74 (9H, m, phe-CH, NCH₂CH₂Cl×2), 3.07 (1H, dd, J_{A,CH}=5.2 Hz, J_{A,B}=13.8 Hz, mel-CH_{2A}), 2.89-2.97 (2H, m, phe-CH_{2A}, mel-CH_{2B}), 2.41 (1H, dd, J_{A,CH}=8.4 Hz, J_{A,B}=13.8 Hz, phe-CH_{2B}).

Mel-L-Glu(OMe)₂ (19)

Boc-melphalan (0.35 g, 0.86 mmol) and L-glutamic acid bis methyl ester (0.20 g, 0.95 mmol, 1.1 eq.) were used to prepare the dipeptide 19 according to the general procedure B. Purification of the final reaction step by flash column chromatography (SiO₂ column, gradient CH₂Cl₂-MeOH 95:5) led to the product as an off-white solid. ES-MS m/z 462.03 [M+H]⁺; UV δₘₐₓ 260, 300 nm.

Mel-D-Glu(OMe)₂ (20)

Boc-melphalan (0.25 g, 0.62 mmol) and D-glutamic acid bis methyl ester (0.15 g, 0.68 mmol, 1.1 eq.) were used to prepare the dipeptide 20 according to the general procedure B. Purification of the final reaction step by flash column chromatography (SiO₂ column, gradient CH₂Cl₂-MeOH 95:5) led to the product as an off-white solid. ES-MS m/z 462.03 [M+H]⁺; UV δₘₐₓ 260, 300 nm.

Glutaryl Cephalosporin bis-DPM Ester (23)

20% NaOH (13 mL, 80 mmol, 2.2 eq.) was added to a suspension of 7-ACA 22 in water (75 mL) giving a dark brown solution. The deacetylation was complete in 30 min by HPLC (0.05% aqueous formic acid-MeCN gradient). Glutaric anhydride (4.7 g, 40 mmol, 1.1 eq.) was added all at once as a solid and the contents stirred for 1.5 h. The reaction was diluted with THF (250 mL) and adjusted to pH 4 with conc. HCl. Freshly prepared diphenyldiazomethane in THF was added via pipette while being monitored by HPLC (5 mM aqueous phosphate pH 7-MeCN gradient). More diphenyldiazomethane was added as the purple color faded from the reaction mixture. Loss of the starting material occurred after about 1-2 h however formation of the second DPM ester was somewhat sluggish. 1 M HCl was added to maintain a pH 4.0-4.5 reaction mixture. After 30 h, the reaction was transferred to a separatory funnel and extracted with EtOAc (1×800 mL, 1×250 mL). The combined organics were dried, filtered and evaporated to an oil that had a white precipitate (benzophenone azine). Addition of EtOAc and cooling caused further precipitation. After filtration, the filtrate was concentrated, taken up in DMSO, and purified by reverse-phase HPLC (C₁₈ column, water-MeCN gradient). The fractions were analyzed by TLC (100% EtOAc) or HPLC. All product containing fractions were pooled and concentrated to a yellow solid that was stored at 0° C. Overall yield: 7.31 g (30%). R_{f} 0.45 (100% EtOAc); ES-MS m/z 699 [M+Na]⁺; UV δₘₐₓ 220, 260 nm. ¹H NMR (DMSO-d₆) δ 8.88 (1H, d, J=8.0 Hz, 7-NH), 7.23-7.52 (20H, m, Ar—H), 6.88 (1H, s, benzhydryl-CH), 6.77 (1H, s, benzhydryl-CH), 5.68 (1H, dd, J_{NH,7}=8.0 Hz, J_{6,7}=4.4 Hz, H-7), 5.16 (1H, bt, J=5.2 Hz, OH), 5.10 (1H, d, J_{6,7}=4.8 Hz, H-6), 4.15-4.23 (1H, m, CH₂OH), 3.62 (1H, d, J_{A,B}=18 Hz, H-2_{A}), 3.54 (1H, d, J_{A,B}=18 Hz, H-2_{B}), 2.46 (2H, t, J=7.6 Hz, glutaryl-CH₂), 2.23 (2H, t, J=7.6 Hz, glutaryl-CH₂), 1.79 (2H, p, J=7.2 Hz, glutaryl-CH₂).

Activated Glutaryl Cephalosporin (24)

The cephalosporin intermediate 23 (3.9 g, 5.76 mmol) was dissolved in methylene chloride (50 mL) followed by the sequential addition of 1,2,2,2-tetrachloroethylchloroformate (0.90 mL, 5.76 mmol) and pyridine (0.52 mL, 6.34 mmol, 1.1 eq.). Reaction was complete according to HPLC in 1 h. Ice (100 g) was added along with 1 M HCl (10 mL) and the contents stirred for 2 min. The mixture was transferred to a separatory funnel that contained 100 mL of CH₂Cl₂ and the layers separated. The organic layer was further washed with water and brine. Drying of the organic phase with MgSO₄ followed by filtration and partial solvent removal in vacuo down to about 100 mL led to a yellow solution that was used in the next step without further purification. R_{f} 0.72 (1:1 hexanes-EtOAc); UV δₘₐₓ 260 nm.

The above mixture was cooled to 0° C. and treated with excess MCPBA (1.81 g, 8.02 mmol, 1.4 eq.). Reaction was complete in 20 min by HPLC analysis. 10% NaHSO₃ (15 mL) followed by 0.1 N NaHCO₃ (100 mL) were added and the contents transferred to a separatory funnel containing chloroform (ca. 50 mL). The aqueous layer was discarded and the organic phase was further washed with 0.1 N NaHCO₃, water, and brine. The organic layer was dried, filtered, and solvent removed to give a tan foam. The product may be purified by flash chromatography (SiO₂, 3:2 to 1:1 hexanes-EtOAc gradient) or used in the next step without further purification. Overall yield (without chromatography): 4.75 g (91% overall). R_{f} 0.30 (1:1 hexanes-EtOAc); ES-MS m/z 923.00 [M+Na]⁺; UV δₘₐₓ 260 nm. ¹H NMR (CDCl₃) δ 7.27-7.48 (20H, m, Ar—H), 6.95 (1H, s, benzhydryl-CH), 6.89 (1H, s, benzhydryl-CH), 6.64 (1H, s, CHCl), 6.60 (1H, d, J=10.0 Hz, 7-NH), 6.13 (1H, dd, J_{NH,7}=10.0 Hz, J_{6,7}=4.8 Hz, H-7), 5.64 (1H, dd, J_{A,B}=14 Hz, J_{CH,A}=5.6 Hz, H-2_{A}), 4.91 (1H, dd, J_{A,B}=14 Hz, J_{CH,A}=5.6 Hz, H-2_{B}), 4.46-4.50 (1H, m, H-6), 3.80 (1H, d, J_{A,B}=18.8 Hz, CH_{2A}OCOO), 3.22 (1H, d, J_{A,B}=19.6 Hz, CH_{2B}OCOO), 2.52 (2H, t, J=7.6 Hz, glutaryl-CH₂), 2.31 (2H, t, J=6.8 Hz, glutaryl-CH₂), 2.02 (2H, p, J=7.2 Hz, glutaryl-CH₂).

Glutaryl C-Mel (26)

A solution of melphalan 2 (0.31 g, 0.97 mmol) in a mixture 0.5 N NaHCO₃ (5.1 mL, 2.53 mmol, 2.6 eq.), water (5 mL), and THF (10 mL) was added to a solution of the activated cephalosporin 24 in THF (25 mL). The solution stirred at ambient temperature for 30 min and was found to be complete at that time by HPLC. Ice (ca. 10 g) and 1 N HCl (5 mL) were added and this mixture was extracted with EtOAc (2×30 mL). The combined organics were washed with brine and dried. Filtration followed by solvent removal led to a yellow film that was taken up in CH₂Cl₂ (100 mL) and treated with anisole (5 mL) and TFA (5 mL). The reaction mixture stood for 1 h. Concentration of the reaction led to a dark oil that was precipitated with ether (ca. 150 mL). The product was filtered off to give a tan solid that was further purified by reverse-phase HPLC (C₁₈ column, 0.1% aqueous TFA-MeCN gradient). The desired fractions were pooled, frozen, and lyophilized to give a fluffy off-white powder. Overall yield: 0.36 g (54%). ES-MS m/z 690.93 [M+H]⁺; UV δₘₐₓ 260, 300 nm. ¹H NMR (DMSO-d₆) δ 8.22 (1H, d, J_{NH,7}=8.0 Hz, mel-NH), 7.65 (1H, d, J=8.0 Hz, 7-NH), 7.06 (2H, d, J=8.8 Hz, mel-Ar—CH×2), 6.63 (2H, d, J=8.8 Hz, mel-Ar—CH×2), 5.75 (1H, dd, J_{NH,7}=8.0 Hz, J_{6,7}=4.8 Hz, H-7), 5.04 (1H, d, J_{A,B}=13.6 Hz, CH_{2A}OCONH), 4.81 (1H, d, J_{6,7}=3.6 Hz, H-6), 4.54 (1H, d, J_{A,B}=13.2 Hz, CH_{2B}OCONH), 3.99-4.07 (1H, m, mel-CH), 3.30-3.75 (10H, m, H-2_{A}, H-2_{B}, NCH₂CH₂Cl×2), 2.85-2.92 (1H, m, mel-CH_{2A}), 2.64-2.73 (1H, m, mel-CH_{2B}), 2.17-2.32 (4H, m, glutaryl-CH₂×2) 1.70 (2H, p, J=7.2 Hz, glutaryl-CH₂).

C-Mel Methyl Ester (27)—General Procedure C

Melphalan methyl ester 3 (95 mg, 0.22 mmol, 1.2 eq.) was suspended in THF (1 mL) followed by the addition of diisopropylethylamine (80 μL, 0.46 mmol, 2.5 eq.) and the activated cephalosporin 25 (0.14 g, 0.18 mmol, 1.0 eq.). The contents stirred for 2 h and was found to be complete by HPLC. The mixture was diluted with ethyl acetate (ca. 20 mL) and the organic layer was successively washed with 10% citric acid, water, saturated sodium bicarbonate, water, and brine. The contents were dried (MgSO₄), filtered, and solvent removed to give the crude ester that was used in the next step without further purification. ES-MS m/z 875.1 [M+H]⁺; UV δₘₐₓ 262 nm. The ester was dissolved in methylene chloride (ca. 10 mL) followed by the addition of anisole (0.39 mL, 3.5 mmol, 20 eq.) and TFA (2 mL). Starting material was gone an hour after standing. Solvent was removed in vacuo to give a dark oil that was subjected to purification by reverse-phase HPLC (C₁₂ column, 0.1% aqueous TFA-MeCN gradient). The desired fractions were pooled and concentrated to give the product as an off-white solid. Yield: 55 mg (41% overall). ES-MS m/z 709.24 [M+H]⁺, 731.22 [M+Na]⁺; UV δₘₐₓ 260, 300 nm.

C-Mel Cyclohexyl Ester (29)

Melphalan cyclohexyl ester (480 mg, 1.24 mmol) and the activated cephalosporin 25 (1.0 g, 1.36 mmol, 1.1 eq.) were dissolved in THF (10 mL) followed by the addition of diisopropylethylamine (0.24 mL, 1.36 mmol, 1.1 eq.). Workup and deprotection was performed as described in general procedure D. Yield: 450 mg (47%). ES-MS m/z 777.30 [M+H]⁺, 775.39 [M−H]⁺; UV δₘₐₓ 260, 300 nm. ¹H NMR (DMSO-d₆) δ 13.82 (1H, br s, COOH), 8.43 (1H, d, J_{NH,7}=8.4 Hz, mel-NH), 7.80 (1H, d, J=8.0 Hz, 7-NH), 7.19-7.32 (5H, m, Ar—H), 7.04 (2H, d, J=8.8 Hz, mel-Ar—CH×2), 6.63 (2H, d, J=8.8 Hz, mel-Ar—CH×2), 5.78 (1H, dd, J_{NH,7}=8.4 Hz, J_{6,7}=4.8 Hz, H-7), 5.06 (1H, d, J_{A,B}=13.2 Hz, CH_{2A}OCONH), 4.81 (1H, d, J_{6,7}=3.6 Hz, H-6), 4.56-4.65 (1H, m, cyclohexyl-CH), 4.55 (1H, d, J_{A,B}=12.8 Hz, CH_{2B}OCONH), 4.02-4.10 (1H, m, mel-CH), 3.30-3.75 (12H, m, H-2_{A}, H-2_{B}, PhCH₂CO, NCH₂CH₂Cl×2), 2.71-2.84 (2H, m, mel-CH₂), 1.15-1.77 (10H, m, cyclohexyl).

C-Mel Cyclobutyl Ester (31)

Melphalan cyclobutyl ester (176 mg, 0.37 mmol) and the activated cephalosporin 25 (275 mg, 0.37 mmol) were dissolved in THF (5 mL) followed by the addition of diisopropylethylamine (130 μL, 0.74 mmol, 2.0 eq.). The reaction was conducted according to general procedure C. Yield: 103 mg (37%). ES-MS m/z 749.1 [M+H]⁺; UV δₘₐₓ 215, 262 nm.

C-Mel t-Butyl Ester (33)

The activated carbonate 25 (0.16 g, 0.22 mmol) and melphalan t-butyl ester 21 (70 mg, 0.22 mmol) were dissolved in THF (5 mL). 1.5 min later diisopropylethylamine (39 μL, 0.22 mmol) was added and the solution was left standing for 30 min. The reaction was diluted with EtOAc (15 mL) and washed with 1 M HCl. The aqueous layer was extracted with EtOAc (2×15 mL) and the combined organics were washed with brine and dried. Filtration of the solution followed by evaporation of the solvent led to a tan solid that was purified by prep-HPLC (C₁₂ column, water-MeCN gradient). Product-containing fractions were pooled and concentrated to a white solid. Yield: 116 mg (59%). UV δₘₐₓ 261, 300 nm.

A solution of the protected cephalosporin intermediate (85 mg, 93 μmol) and anisole (0.2 mL, 1.9 mmol, 20 eq.) in CH₂Cl₂ (10 mL, 0.1 M solution) had added TFA (100 μL, 1.3 mmol, ca. 14 eq, 1% solution). The reaction was monitored by HPLC and found to be complete in 8 h. Solvent was removed in vacuo and the reaction mixture immediately purified by reverse-phase HPLC (C₁₂ column, 0.1% aqueous TFA-MeCN gradient). The desired product was isolated as a white solid. Yield: 47 mg (68%). ES-MS m/z 751.21 [M+H]⁺; UV δₘₐₓ 260, 300 nm.

Glutaryl Cephalosporin (26)

A suspension of 7-ACA 22 (5.0 g, 18.2 mmol) in water (50 mL) at 0° C. had added to it 20% aqueous NaOH (6.4 mL, 40 mmol, 2.2 eq.) while stirring. The resulting dark brown solution continued stirring for 30 min. At this point no starting material was detected by HPLC analysis. Glutaric anhydride (2.35 g, 20 mmol, 1.1 eq.) was added as a solid in one portion and the reaction mixture was maintained at pH 8.5-9.0 with Et₃N. After 1.5 h, the mixture was concentrated and purified by preparative-HPLC (C₁₈ column, 0.1% aqueous Et₃N/CO₂-MeCN gradient). The product was isolated as a brown foam upon extensive drying of the desired fractions. Yield: 7.65 g (76%). UV δₘₐₓ 202, 260 nm.

Glutaryl C-Mel Cyclohexyl Ester (30)—General Procedure D

The trifluoroacetate salt of melphalan cyclohexyl ester 5 (22 mg, 43 μmol) and the activated glutaryl cephalosporin 24 (39 mg, 43 μmol) were dissolved in THF (2 mL) followed by the addition of diisopropylethylamine (15.1 μL, 86 μmol, 2.0 eq.). The contents stirred while being monitored by HPLC. The mixture was purified by reverse-phase HPLC (C₁₈ column, water-MeCN gradient) and the product was isolated as a white solid powder. R_{f} 0.15 (1:1 hexanes-EtOAc); ES-MS m/z 1128 [M+Na]⁺.

The intermediate was taken up in CH₂Cl₂ (10 mL) and to this was added anisole (96 μL, 0.88 mmol, 20 eq.) and TFA (2 mL, 20% v/v). No starting material was detected after 30 min according to HPLC analysis. The reaction solvent was removed to give a brown oil that was placed under high vacuum. The residue was taken up in the minimum amount of DMSO and purified by reverse-phase HPLC (C₁₈ column, 0.1% formic acid in a water-MeCN gradient). The desired fractions were pooled and concentrated in vacuo to give the product as an off-white solid. Yield: 22.5 mg (66%). ES-MS m/z 773 [M+H]⁺; UV δₘₐₓ 200, 262 nm. ¹H NMR (DMSO-d₆) δ 8.23 (1H, d, J_{NH,7}=8.0 Hz, mel-NH), 7.80 (1H, d, J=8.0 Hz, 7-NH), 7.04 (2H, d, J=8.8 Hz, mel-Ar—CH×2), 6.63 (2H, d, J=8.8 Hz, mel-Ar—CH×2), 5.76 (1H, dd, J_{NH,7}=8.0 Hz, J_{6,7}=4.8 Hz, H-7), 5.06 (1H, d, J_{A,B}=13.6 Hz, CH_{2A}OCONH), 4.83 (1H, d, J_{6,7}=3.6 Hz, H-6), 4.56-4.65 (1H, m, cyclohexyl-CH), 4.55 (1H, d, J_{A,B}=13.6 Hz, CH_{2B}OCONH), 4.02-4.10 (1H, m, mel-CH), 3.30-3.75 (10H, m, H-2_{A}, H-2_{B}, NCH₂CH₂Cl×2), 2.71-2.84 (2H, m, mel-CH₂), 2.18-2.33 (4H, m, glutaryl CH₂×2), 1.70 (2H, p, J=7.6 Hz, glutaryl CH₂), 1.15-1.66 (10H, m, cyclohexyl).

Glutaryl C-Mel Cyclobutyl Ester (32)

The melphalan cyclobutyl ester (0.10 g, 0.29 mmol) and the activated glutaryl cephalosporin 24 (0.26 g, 0.29 mmol) were dissolved in THF (3 mL) followed by the addition of diisopropylethylamine (56 μL, 86 μmol, 1.1 eq.). The reaction was performed according to general procedure D. Reaction was complete in 6 h. Ice was added followed by 1 M HCl and EtOAc. The layers were separated and the organic phase was further washed with water and brine and dried (MgSO₄). Removal of solvent gave the product as a tan residue that was used without further purification.

The intermediate was taken up in CH₂Cl₂ (10 mL) and to this was added anisole (0.61 mL, 5.6 mmol, 20 eq.) and TFA (3 mL). No starting material was detected after 1 h according to HPLC analysis. The reaction mixture was concentrated, taken up in the minimum amount of DMSO and purified by reverse-phase HPLC (C₁₈ column, 0.1% formic acid in a water-MeCN gradient). The desired fractions were pooled, frozen, and lyophilized to give a flaky light yellow solid as the final product. Yield: 123 mg (59% overall). ES-MS m/z 745 [M+H]⁺; UV δₘₐₓ 200, 262 nm.

Glutaryl C-Mel-L-Phe-NH₂ (35)

A solution of Mel-L-Phe-NH₂ 17 (0.59 g, 1.3 mmol) and the activated glutaryl cephalosporin 24 (1.17 g, 1.3 mmol) in THF (30 mL) had added to it diisopropylethylamine (0.23 mL, 1.3 mmol, 1.0 eq.). After 10 min, an addition 1.0 eq. of diisopropylethylamine was added. Workup and deprotection was performed according to general procedure D. The product was purified by reverse-phase HPLC (C₁₂ column, 0.1% formic acid in a water-MeCN gradient). The desired fractions were pooled, frozen, and lyophilized to give a white powdered solid as the final product. Yield: 0.42 g (39% overall). ES-MS m/z 836.85 [M+H]⁺, 858.83 [M+Na]⁺; UV δₘₐₓ 260, 300 nm. ¹H NMR (DMSO-d₆) δ 8.21 (1H, d, J_{NH,7}=8.0 Hz, mel-NH), 7.97 (1H, d, J=8.0 Hz, 7-NH), 7.45 (1H, d, J=8.0 Hz, phe-NH), 7.12-7.28 (5H, m, phe-Ar—CH×5), 7.02 (2H, d, J=8.8 Hz, mel-Ar—CH×2), 6.59 (2H, d, J=8.8 Hz, mel-Ar—CH×2), 5.76 (1H, dd, J_{NH,7}=8.0 Hz, J_{6,7}=4.8 Hz, H-7), 5.02 (1H, d, J_{A,B}=13.6 Hz, CH_{2A}OCONH), 4.77 (1H, d, J_{6,7}=3.6 Hz, H-6), 4.53 (1H, d, J_{A,B}=13.6 Hz, CH_{2B}OCONH), 4.39-4.47 (1H, m, mel-CH), 4.02-4.10 (1H, m, phe-CH), 3.21-3.72 (10H, m, H-2_{A}, H-2_{B}, NCH₂CH₂Cl×2), 3.00 (1H, dd, J=4.4 Hz, J=13.8 Hz, mel-CH_{2A}), 2.71-2.84 (2H, m, phe-CH_{2A}, mel-CH_{2B}), 2.47-2.55 (1H, m, phe-CH_{2A}), 2.18-2.32 (4H, m, glutaryl-CH₂×2) 1.71 (2H, p, J=7.2 Hz, glutaryl-CH₂).

Glutaryl C-Mel-D-Phe-NH₂ (36)

A solution of Mel-D-Phe-NH₂ 18 (0.11 g, 0.24 mmol) and the activated glutaryl cephalosporin 24 (0.22 g, 0.24 mmol) in THF (30 ml) had added to it diisopropylethylamine (43 μl, 0.24 mmol). After 10 min, an addition 1.0 eq. Of diisopropylethylamine was added. Workup and deprotection was performed according to general procedure D. The product was purified by reverse-phase HPLC (C₁₂ column, 0.1% formic acid in a water-MeCN gradient). The desired fractions were pooled, frozen, and lyophilized to give a white powdered solid as the final product. Yield: 112 mg (55% overall). ES-MS m/z 836.88 [M+H]⁺, 858.86 [M+Na]⁺; UV δₘₐₓ 260, 300 nm.

The invention is further described in the following examples, which are not intended to limit the scope of the invention.

### EXAMPLE 1

In Vitro Cytotoxicity Assay. The H3677 melanoma cell line was seeded at a density of 2×10³ per well in a 96 well plate and allowed to adhere overnight in RPMI 1640 medium containing 10% FBS in the absence of antibiotics. IC₅₀ values are based on a 4 hr exposure of the prodrug to H3677 melanoma cells in the presence or absence of either 50 ng/mL of β-lactamase (BL) or L49-beta-lactamase followed by washing of the cells and 96 hr incubation at 37° C. Also using the H3677 cell line as described above, IC₅₀ values were also determined for the drug alone, as shown below, however without the presence or absence of either 50 ng/mL of □-lactamase (BL) or L49-beta-lactamase. Alamar Blue (Biosource International, Camarillo, Calif.) was added to 10% of the total culture volume. Cells were incubated for 4 h and dye reduction was measured on a Fusion HT fluorescent plate reader (Packard Instruments, Meriden, Conn.).

| Drug | IC₅₀ (μM) |
|---|---|
| melphalan | 2.8 |
| melphalan methyl ester | 0.054 |
| melphalan t-butyl ester | 3.34 |
| melphalan cyclohexyl ester | 0.074 |
| melphalan phenethyl ester | 0.17 |
| melphalan cyclobutyl ester | 0.2 |
| melphalan (2-methyl)phenethyl ester | 0.08 |
| melphalan cyclooctyl ester | 5 |
| melphalan menthyl ester | 28 |
| melphalan (2-trifluoromethyl)phenethyl ester | 3 |
| melphalan cis-4-t-butylcyclohexyl ester | 13 |
| melphalan (2-methyl)phenethyl ester | 2.4 |
| melphalan (4-butyl)phenethyl ester | 4.6 |
| melphalan trans-4-t-butylcyclohexyl ester | 3.6 |
| Mel-L-Phe-ol | 1.7 |
| Mel-L-Phe-NH2 | 0.03 |
| Mel-phenethyl amide | 0.97 |
| Mel-Gly-nitrophenyl amide | 1.01 |
| Mel-L-phenylglycine amide | 0.38 |
| Mel-cyclohexyl amide | 1.57 |
| Mel-L-Met amide | 0.4 |
| Mel-(1S,2R)-norephedrine | 0.02 |
| Mel-L-Val-NH2 | 0.37 |
| Mel-L-Glu bis methyl ester | 1.89 |
| Mel-D-Glu bis methyl ester | 3.95 |
| Mel-L-Phe-NH2 | 0.04 |
| Mel-D-Phe-NH2 | 2.68 |
| Mel-L-Phe | 7.5 |
| Mel-L-Phe-L-Phe-NH2 | 0.025 |
| Mel-L-Phe cyclohexyl amide | 0.015 |
| Mel-L-Ser(OBz)-NH2 | 0.1 |
| Mel-L-homoPhe-NH2 | 0.09 |
| melphalan anilide | 1.2 |
| melphalan amide | 0.64 |
| Mel-propargylalanine-NH2 | 0.31 |
| C-Mel-L-Phe | >20 |

| | IC₅₀ (μM) | IC₅₀ (μM) | IC₅₀ (μM) |
|---|---|---|---|
| Prodrug | without βL | with βL | with L49-βL |
| C-Mel | 47.8 | 1.9 | 2.3 |
| C-Mel methyl ester | 0.65 | 0.0018 | |
| C-Mel t-butyl ester | 32.3 | 2.1 | |
| C-Mel cyclohexyl ester | 1.6 | 0.083 | 0.23 |
| glutaryl C-Mel cyclohexyl ester | 1.86 | 0.087 | 0.14 |
| C-Mel cyclobutyl ester | 10 | 0.8 | |
| glutaryl C-Mel | 26.3 | 1.4 | 2.4 |
| glutaryl C-Mel t-butyl ester | 25 | 2.3 | |
| glutaryl C-Mel-L-Phe-NH2 | 3.6 | 0.05 | |
| glutaryl C-Mel-D-Phe-NH2 | 30 | 2.1 | |
| C-Mel-L-Phe | >20 | 2.47 | 3.9 |

The discussion above is descriptive, illustrative and exemplary and is not to be taken as limiting the scope defined by any appended claims. Various references, including patent applications, patents, and scientific publications, are cited herein, the disclosures of each of which is incorporated herein by reference in its entirety.

1. A method for the delivery of a cytotoxic agent to tumor cells comprising:
administering an effective amount of at least one antibody-enzyme conjugate comprising an antibody reactive with an antigen on the surface of the tumor cells conjugated to an enzyme which converts at least one prodrug having the formula 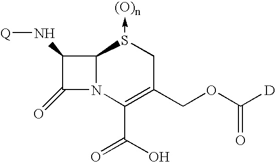
wherein
Q=H or salt thereof, C═O-alkyl, C═O-PEG, C═O-cycloalkyl, C═O-aryl, C═O-arylalkyl, CO₂R, or CONRR′ where R and R′ are, independently, an alkyl, alkenyl, alkynyl, heteroaryl alkyl, substituted alkyl, substituted aryl, substituted arylalkyl, heteroaryl, PEG, cycloalkyl, aryl, or arylalkyl;
n=0, 1, or 2;
and D having the formula: 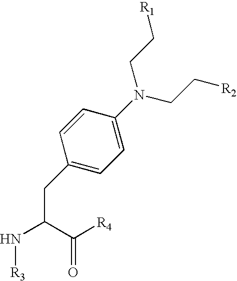
where R₁, R₂=independently halogens, O-mesylate, or O-tosylate,
R₃=H or lower alkyl groups C₁-C₆,
R₄=OH, PEG, —NH₂, NHR, NRR′, where R and R′ are, independently, alkyl, alkenyl, alkynyl, heteroaryl alkyl, substituted alkyl, substituted aryl, substituted arylalkyl, heteroaryl, or is comprised of a peptide as follows: 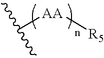
where AA is any given amino acid,
n=1 to 12 and
R₅ represents the manner in which the C-terminal amino acid is capped at the carboxy terminus, if at all, and a pharmaceutically acceptable salt or solvent thereof, that is weakly cytotoxic to tumor cells compared to its corresponding parent drug, into the more cytotoxic parent drug.

2. The method of claim 1 wherein Q is a C═O-alkyl.

3. The method of claim 2 wherein the C═O-alkyl is a glutaryl moiety.

4. The method of claim 1 wherein R₄ is

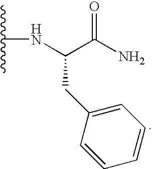

5. The method of claim 1 wherein R₄ is

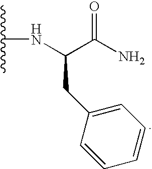

6. The method of claim 1 wherein R₄ is

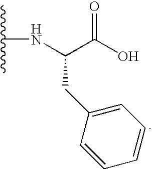

7. The method of claim 1 wherein R₄ is

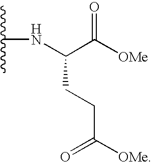

8. The method of claim 1 wherein R₄ is

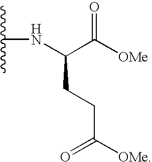

9. The method of claim 1 where Q is a C═OR where R is

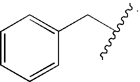

10. The method of claim 1 where -AA- is natural or synthetic, D or L, R or S, essential or non essential, alpha amino acids, beta amino acids, 3-amino acids, 4-amino acids, and 5-amino acids.

11. The method of claim 1 wherein D is a nitrogen mustard compound.

12. The method of claim 11 wherein the nitrogen mustard compound is chlorambucil, phenylacetic mustard, phenylproprionic mustard, and melphalan.

13. The method of claim 1, wherein the antibody is selected from the group consisting of polyclonal, monoclonal or chimeric antibodies.

14. The method of claim 1, wherein the antibody is monoclonal antibody L49.

15. The method of claim 1, wherein the enzyme is a beta-lactamase.

16. The method of claim 1, wherein the parent drug is selected from the group consisting of melphalan and other nitrogen mustards.

17. The method of claim 1, wherein the parent drug is melphalan.

18. The method of claim 1, wherein the antibody-enzyme conjugate is L49-beta-lactamase.

19. The method of claim 1, wherein the tumor cells are of an origin selected from the group consisting of carcinomas, melanomas, and lymphomas.

20. The method of claim 1 wherein Q is 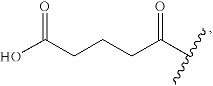
n=1, and D is melphalan.

21. The method of claim 1 wherein Q is 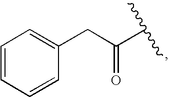
n=1, and D is melphalan.

22. A method for the prevention or treatment of cancer, an immune disorder, or an infectious disease comprising
administering to a subject an effective amount of compound of the formula: 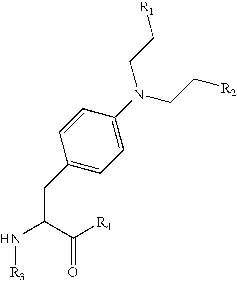
where R₁, R₂=independently halogens, O-mesylate, or O-tosylate,
R₃=H or lower alkyl groups C₁-C₆,
R₄=OH, PEG, —NH₂, NHR, NRR′, where R and R′ are, independently, alkyl, alkenyl, alkynyl, heteroaryl alkyl, substituted alkyl, substituted aryl, substituted arylalkyl, heteroaryl, or is comprised of a peptide as follows: 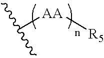
where AA is any given amino acid,
n=1 to 5 and
R₅ represents the manner in which the C-terminal amino acid is capped at the carboxy terminus, if at all, and a pharmaceutically acceptable salt or solvent thereof.

23. The method of claim 22 wherein R₄ is

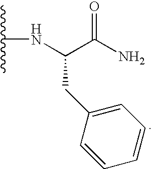

24. The method of claim 22 wherein R₄ is

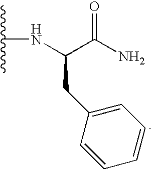

25. The method of claim 22 wherein R₄ is

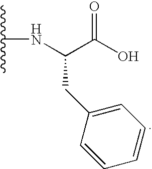

26. The method of claim 22 wherein R₄ is

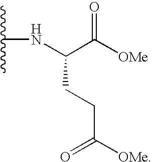

27. The method of claim 22 wherein R₄ is

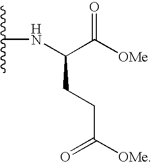

28. The method of claim 22 wherein -AA- is natural or synthetic, D or L, R or S, essential or non essential, alpha amino acids, beta amino acids, 3-amino acids, 4-amino acids, and 5-amino acids.

29. The method of claim 22 wherein D is a nitrogen mustard compound.

30. The method of claim 29 wherein the nitrogen mustard compound is chlorambucil, phenylacetic mustard, phynelproprionic mustard, and melphalan.

31. A prodrug comprising an enzyme substrate portion and a drug unit, the drug unit having the formula: 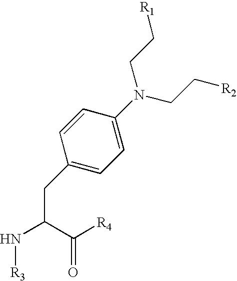
where R₁, R₂=independently halogens, O-mesylate, or O-tosylate,
R₃=H or lower alkyl groups C₁-C₆,
R₄=OH, PEG, —NH₂, NHR, NRR′, where R and R′ are, independently, alkyl, alkenyl, alkynyl, heteroaryl alkyl, substituted alkyl, substituted aryl, substituted arylalkyl, heteroaryl, or is comprised of a peptide as follows: 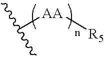
where AA is any given amino acid,
n=1 to 5 and
R₅ represents the manner in which the C-terminal amino acid is capped at the carboxy terminus, if at all, and a pharmaceutically acceptable salt or solvent thereof.

32. The prodrug of claim 31 wherein R₄ is

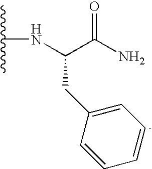

33. The prodrug of claim 31 wherein R₄ is

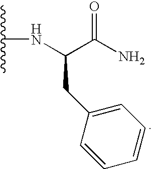

34. The prodrug of claim 31 wherein R₄ is

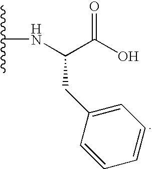

35. The prodrug of claim 31 wherein R₄ is

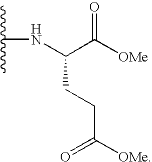

36. The prodrug of claim 31 wherein R₄ is

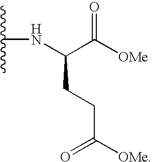

37. The prodrug of claim 31 wherein -AA- is natural or synthetic, D or L, R or S, essential or non essential, alpha amino acids, beta amino acids, 3-amino acids, 4-amino acids, and 5-amino acids.

38. The prodrug of claim 31 wherein D is a nitrogen mustard compound.

39. The prodrug of claim 38 wherein the nitrogen mustard compound is chlorambucil, phenylacetic mustard, phenylproprionic mustard, and melphalan.

40. The prodrug of claim 31 wherein the enzyme substrate portion is a beta-lactam.

41. The prodrug of claim 40 wherein the beta-lactam has the formula: 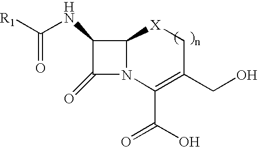
wherein X=CH₂, CHR, O, S, SO, or SO₂ and R is a C₁-C₆ alkyl,
n=0, 1; and
wherein R₁ includes:

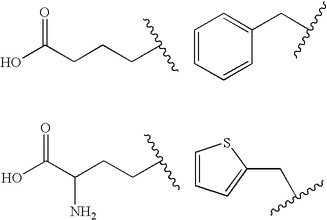

42. The prodrug of claim 40 wherein the beta-lactam is cephalosporin.

43. A prodrug having the formula 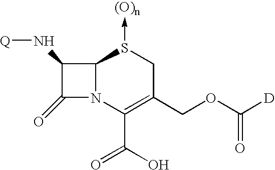
wherein
Q=H or salt thereof, C═O-alkyl, C═O-PEG, C═O-cycloalkyl, C═O-aryl, C═O-arylalkyl, CO₂R, or CONRR′ where R and R′ are, independently, an alkyl, alkenyl, alkynyl, heteroaryl alkyl, substituted alkyl, substituted aryl, substituted arylalkyl, heteroaryl, PEG, cycloalkyl, aryl, or arylalkyl;
n=0, 1, or 2;
and D having the formula: 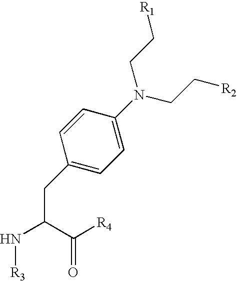
where R₁, R₂=independently halogens, O-mesylate, or O-tosylate,
R₃=H or lower alkyl groups C₁-C₆,
R₄=OH, PEG, —NH₂, NHR, NRR′, where R and R′ are, independently, alkyl, alkenyl, alkynyl, heteroaryl alkyl, substituted alkyl, substituted aryl, substituted arylalkyl, heteroaryl, or is comprised of a peptide as follows: 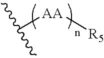
where AA is any given amino acid,
n=1 to 5 and
R₅ represents the manner in which the C-terminal amino acid is capped at the carboxy terminus, if at all, and a pharmaceutically acceptable salt or solvent thereof.

44. The prodrug of claim 43 wherein Q is a C═O-alkyl.

45. The prodrug of claim 44 wherein the C═O-alkyl is a glutaryl moiety.

46. The prodrug of claim 43 wherein R₄ is

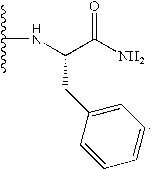

47. The prodrug of claim 43 wherein R₄ is

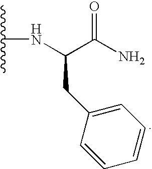

48. The prodrug of claim 43 wherein R₄ is

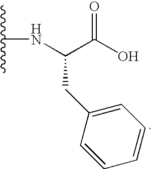

49. The prodrug of claim 43 wherein R₄ is

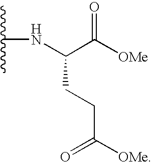

50. The prodrug of claim 43 wherein R₄ is

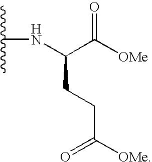

51. The prodrug of claim 43 where Q is a C═OR where R is

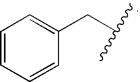

52. The prodrug of claim 43 where -AA- is natural or synthetic, D or L, R or S, essential or non essential, alpha amino acids, beta amino acids, 3-amino acids, 4-amino acids, and 5-amino acids.

53. The prodrug of claim 43 wherein D is a nitrogen mustard compound.

54. The prodrug of claim 53 wherein the nitrogen mustard compound is chlorambucil, phenylacetic mustard, phynelproprionic mustard, and melphalan.

55. The prodrug of claim 43 wherein Q is a glutaryl moiety and D is melphalan.

56. The prodrug of claim 43 wherein Q is H and D is melphalan.

57. A compound having the formula having the formula: 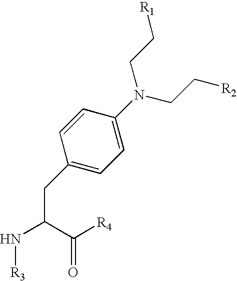
where R₁, R₂=independently halogens, O-mesylate, or O-tosylate,
R₃=H or lower alkyl groups C₁-C₆,
R₄=OH, PEG, —NH₂, NHR, NRR′, where R and R′ are, independently, alkyl, alkenyl, alkynyl, heteroaryl alkyl, substituted alkyl, substituted aryl, substituted arylalkyl, heteroaryl, or is comprised of a peptide as follows: 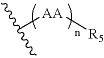
where AA is any given amino acid,
n=1 to 5 and
R₅ represents the manner in which the C-terminal amino acid is capped at the carboxy terminus, if at all, and a pharmaceutically acceptable salt or solvent thereof.

58. The compound of claim 57 wherein R₄ is

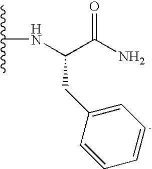

59. The compound of claim 57 wherein R₄ is

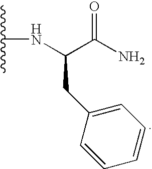

60. The compound of claim 57 wherein R₄ is

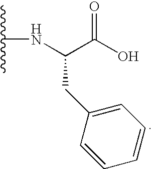

61. The compound of claim 57 wherein R₄ is

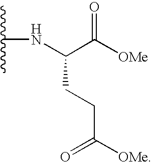

62. The compound of claim 57 wherein R₄ is

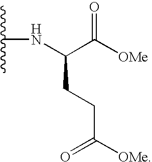

63. The compound of claim 57 where -AA- is natural or synthetic, D or L, R or S, essential or non essential, alpha amino acids, beta amino acids, 3-amino acids, 4-amino acids, and 5-amino acids.

64. A pharmaceutical composition comprising a pharmaceutically effective amount of a prodrug having the formula 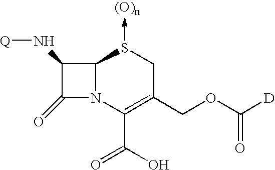
wherein
Q=H or salt thereof, C═O-alkyl, C═O-PEG, C═O-cycloalkyl, C═O-aryl, C═O-arylalkyl, CO₂R, or CONRR′ where R and R′ are, independently, an alkyl, alkenyl, alkynyl, heteroaryl alkyl, substituted alkyl, substituted aryl, substituted arylalkyl, heteroaryl, PEG, cycloalkyl, aryl, or arylalkyl;
n=0, 1, or 2;
and D having the formula: 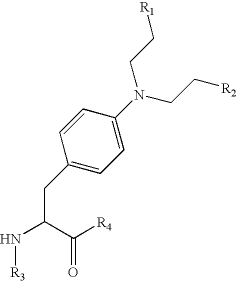
where R₁, R₂=independently halogens, O-mesylate, or O-tosylate,
R₃=H or lower alkyl groups C₁-C₆,
R₄=OH, PEG, —NH₂, NHR, NRR′, where R and R′ are, independently, alkyl, alkenyl, alkynyl, heteroaryl alkyl, substituted alkyl, substituted aryl, substituted arylalkyl, heteroaryl, or is comprised of a peptide as follows: 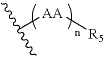
where AA is any given amino acid,
n=1 to 5 and
R₅ represents the manner in which the C-terminal amino acid is capped at the carboxy terminus, if at all, and a pharmaceutically acceptable salt or solvent thereof, in admixture with a pharmaceutically acceptable carrier, diluent or excipient.

65. The pharmaceutical composition of claim 64 wherein Q is a glutaryl moiety and D is melphalan.

66. The pharmaceutical composition of claim 64 wherein Q is H and D is melphalan.
